(19) United States
(12) Patent Application Publication
(10) Pub. No.: US 2003/0041341 A1
(43) Pub. Date: Feb. 27, 2003

(54) Non-human transgenic animal whose germ cells and somatic cells contain a knockout mutation in DNA encoding 4E-BP1

(76) Inventors: Nahum Sonenberg, Cote St-Luc (CA); Michel Tremblay, Dorval (CA); Kyoko Tsukiayama-Kohara, Kita-Ku (JP)

Correspondence Address: MORRISON & FOERSTER LLP, 755 PAGE MILL RD, PALO ALTO, CA, 94304-1018, US

(21) Appl. No.: 09/973,473

(22) Filed: Oct. 9, 2001

Related U.S. Application Data
(60) Provisional application No. 60/128,559, filed on Apr. 9, 1999.
(60) Provisional application No. 60/179,743, filed on Feb. 2, 2000.

(30) Foreign Application Priority Data
Apr. 7, 2000 (CA) PCT/CA00/00388

(51) Int. Cl. A01K 67/027; C12N 5/06

(52) U.S. Cl. 800/18; 435/354; 800/21

(57) ABSTRACT

The eukaryotic mRNA 5′ cap structure is recognized by eIF4E, which plays an essential role in translational control and cell growth. Members of a family of proteins called eIF4E-binding proteins (4E-BPs) inhibit the activity of eIF4E and consequently repress translation. Following exposure of cells to hormones, cytokines and growth factors, 4E-BPs become hyperphosphorylated and dissociate from eIF4E, to relieve translation inhibition. The phosphorylation events leading to 4E-BP1 dissociation from eIF4E are mediated by the P13-kinase/FRAP/mTOR signaling pathway. The present study addresses the biological importance of 4E-BP1 in vivo by disrupting its gene in the mouse. Homozygous 4E-BP1 deficient mice are healthy and develop normally. However, they show an important decrease in white adipose tissue and blood glucose level, and the males show a decrease in total body weight and an increase in resting metabolic rate. Primary mouse embryo fibroblasts show accelerated cell growth and enhanced cap-dependent translation, coincident with an increase in eIF4E phosphorylation.

### FIELD OF THE INVENTION

The present invention relates to a non-human transgenic animal whose germ cells and somatic cells contain a knockout mutation in DNA encoding 4E-BP1. More particularly the present invention relates to a non-human transgenic mammal whose germ cells and somatic cells contain a knockout mutation in DNA encoding 4E-BP1 and more specifically to transgenic mice whose germ cells and somatic cells contain a knockout mutation in DNA encoding 4E-BP1. In one particular embodiment, mice containing a disruption of both copies of the 4E-BP1 gene lack a detectable expression of the 4E-BP1 protein. Until the present invention, the interaction between 4E-BP1 and eIF-4E and their effect on homeostasis, fat tissue growth, glucose metabolism, had not been identified. The present invention also relates to assays and methods to identify and select agents which modulate eIF-4E sequestration and particularly the activity of 4E-BP1.

### BACKGROUND OF THE INVENTION

Obesity is a prevalent disorder that often leads to diabetes, cardiovascular disease, and joint disorders Although the precise mechanism which leads to the development of obesity has yet to be precisely determined, it appears clear that a number of mechanisms, which normally function to maintain homeostasis and normal body weight are involved.

Eukaryotic mRNA translation initiation is an exquisitely regulated process involving assembly of a large multiprotein-RNA complex that directs ribosomes to the initiation codon. In the most general case of cap-dependent translation, protein synthesis begins with 7-methyl-G(5′)ppp(5′)N recognition by eukaryotic initiation factor 4F (eIF-4F). In higher eukaryotes, eIF-4F consists of three polypeptide chains: eIF-4E, eIF-4A, and eIF-4G (reviewed in (Sonenberg, 1996). eIF-4E is a 25 kDa protein that specifically interacts with the cap structure. eIF-4A is an ATP-dependent, RNA helicase, which in concert with another general translation initiation factor (eIF-4B) is thought to unwind the 5′ untranslated region of the mRNA. Mammals possess two isoforms of eIF-4G, eIF-4GI and eIF-4GII (171 kDa and 176 kDa, respectively), which are 46% identical at the amino acid level. Both eIF-4GI and eIF-4GII act as molecular bridges between eIF-4E and eIF-4A, yielding eIF-4F. The eIF-4Gs also interact with eIF-3, a multisubunit translation initiation factor associated with the 40S ribosomal subunit, enabling eIF-4F to recruit the 40S ribosomal subunit to the 5′ end of the mRNA. In addition, eIF-4GI and eIF-4GII can bind directly to the poly(A) binding protein (PABP). Association of eIF-4G and PABP is correlated with eIF-4G-induced circularization of the mRNA in presence of eIF-4E (Wells et al., 1998). The biological significance of mRNA circularization is unknown at present, but is thought to enhance translation efficiency perhaps by facilitating ribosome recycling (reviewed in Jacobson, 1996).

As a central player in translation initiation, eIF-4G is a logical target for regulation of cellular protein expression. Mammalian 4E-BP1, 4E-BP2, 4E-BP3 (reviewed in Sonenberg, 1996) and yeast p20 (Altmann et al., 1997) inhibit cap-dependent protein synthesis by competing with eIF-4G for binding to eIF-4E. Biochemical studies have demonstrated that eIF-4G and the 4E-BPs occupy mutually-exclusive binding sites on the surface of eIF-4E (Haghighat et al., 1995), thereby blocking assembly of the translation machinery without affecting cap recognition. Sequence analyses of the 4E-BPs and the eIF-4Gs suggest that these two protein families have converged on the same eIF-4E binding strategy, which employs a Tyr-X-X-X-X-Leu-φ eIF4E-recognition motif (where X is variable and φ is a hydrophobic amino acid, and more particularly Leu, Met, or Phe) (Mader et al., 1995; Altmann et al., 1997). Treatment of cells with mitogens or growth factors upregulates cap-dependent translation, at least in part, by relieving the repressive effects of the 4E-BPs. After phosphorylation of one or more serine and/or threonine residues by the phosphatidylinositol 3-kinase signal transduction pathway, the 4E-BPs are no longer able bind to eIF-4E allowing translation initiation to proceed (reviewed in Sonenberg and Gingras, 1998).

The structures of mammalian (Marcotrigiano et al., 1997) and yeast (Matsuo et al., 1997) eIF-4E bound to the cap analog 7-methyl-GDP resemble a cupped hand, consisting of a curved, 8-stranded antiparallel β-sheet, backed by three long α-helices. The cap analog binds in a narrow slot on the molecule's concave surface. 7-methyl-guanine recognition by eIF-4E is mediated by π-π stacking between two conserved tryptophans and three Watson-Crick-like hydrogen bonds, involving a backbone amino group and the side chain of a conserved glutamate. The methyl group makes a van der Waals contact with a third conserved tryptophan. On its convex dorsal surface, eIF-4E displays a phylogenetically-invariant hydrophobic/acidic portion (see FIG. 5B in Marcotrigiano et al., 1997) that was predicted to be the binding site for the Tyr-X-X-X-X-Leu-φ motifs of both eIF-4G and the 4E-BPs. This assertion has been partially confirmed by the results of NMR experiments using yeast eIF-4E and mammalian 4E-BP1 (Fletcher et al., 1998).

More recently, two high-resolution crystal structures of binary complexes of eIF-4E plus 7-methyl-GDP interacting with eIF-4E-recognition motifs from mammalian eIF-4GII (referred to as the active complex) and 4E-BP1 (referred to as the inhibited complex) were described (Marcotrigiano et al., 1999, Molecular Cell 3:707-716). Therein, it was shown that both oligopeptides bind the same conserved portion of eIF-4E's convex dorsal surface, far from the cap-binding slot. The two Tyr-X-X-X-X-Leu-φ motifs adopt identical L-shaped, extended chain/a-helical conformations, stabilized by similar contacts within each peptide and with eIF-4E. Biochemical studies of full-length 4E-BP1 and the two oligopeptides document that they bind eIF-4E with similar affinities, lack secondary structure in the absence of eIF-4E, and inhibit translation in vitro. It was suggested that 4E-BP1 is a molecular mimic of eIF-4G, that undergoes the same disorder-to-order transition on binding to eIF-4E. The resulting competition permits regulation of translation initiation in eukaryotes, which can be overcome by phosphorylation of the 4E-BPs (Marcotrigiano et al., 1999, supra).

Methods for screening agents useful in treating hormone disorders and especially diabetes have been disclosed in U.S. Pat. No. 5,874,231. More particularly, U.S. Pat. No. 5,874,231 teaches methods for identifying agents that mimic the activity of a hormone in modulating the interaction between eIF-4E and 4E-BP1 and between eIF-4E and 4E-BP2. A number of assays and methods are also described therein.

There nevertheless remains a need to better identify which homeostatic mechanism, when disrupted or malfunctioning is implicated in fat tissue growth and the development of obesity and related diseases (i.e. diabetes). In addition, there remains a need to provide animal models of obesity and related diseases, and model systems which can enable the identification and selection of agents which modulate the pathways implicated in fat tissue growth and the development of obesity and related diseases. Furthermore, there remains a need to identify a target for the eventual therapy of obesity and related diseases.

The present invention seeks to meet these and other needs. Furthermore, non-human transgenic animals of the present invention are useful in helping to meet these and other needs.

The present description refers to a number of documents, the content of which is herein incorporated by reference in their entirety.

### SUMMARY OF THE INVENTION

In general, the present invention relates to non-human transgenic animals that seek to overcome the drawbacks of the prior art and seek to provide screening assays and agents identified by same which can modulate pathways implicated in fat tissue growth and metabolism and energy homeostasis.

Broadly, the invention relates to 4E-BP1-deficient non-human transgenic animals and more particularly to transgenic mammals. More specifically, the invention relates to a transgenic non-human mammal whose germ cells and somatic cells contain a knockout mutation in DNA encoding the 4E-BP1 polypeptide. In one embodiment, the transgenic mammal also includes germ cells and somatic cells expressing DNA encoding a non-endogenous 4E-BP1 polypeptide. In an especially preferred embodiment, the transgenic mammal also includes germ cells and somatic cells expressing DNA encoding a human 4E-BP1 polypeptide.

Also in general, the present invention relates to the surprising demonstration that the 4E-BP1 and eIF-4E interaction impacts fat metabolism. Indeed, the 4E-BP1 knockout mouse of the present invention displays changes in fat tissue growth, metabolism, glucose metabolism, and weight gain. It is therefore the aim of the present invention to provide the means to affect these processes. The effect of the disruption of 4E-BP1 in the knockout mice of the present invention demonstrates that an alteration of 4E-BP1 activity, or of its partner eIF-4E, can modulate fat tissue growth, metabolism and more particularly glucose metabolism in vivo (i.e. in a living animal). In view of the hypoglycemia observed in the knockout mice of the present invention, it appears that the 4E-BP1 knockout can modulate insulin signalling in a living animal. The knockout mice of the present invention also demonstrate that the alteration of the activity of 4E-BP1 (or eIF-4E, indirectly) can affect weight gain in an animal.

Based on the results presented herein, the inhibition of 4E-BP1 is relevant to the treatment of non-insulin dependent diabetes (type II diabetes) as well as obesity.

Until the present invention, studies on eIF-4E and 4E-BP1 interactions were limited to in vitro studies and studies in cultured cells, or extracts thereof. Therefore, such studies did not assess the action of 4E-BP1 on eIF-4E and on metabolic pathways dependent on such interactions, or indirectly on the desequestration of eIF-4E by a negative regulator of cap-dependent translation, which could result in a physiologically significant effect such as, for example, fat tissue growth, metabolism, glucose metabolism, or weight gain in a living animal or preferably in a living mammal.

Because of the complexity of translation regulation and of the essentiality of eIF-4E for the maintenance of homeostasy in cells and animals, there was a need for the study of the interaction between eIF-4E and 4E-BP1 (or a sequestering of eIF-4E; e.g. an inhibited complex) in an environment which is as close to the living situation as possible. The transgenic animals of the present invention provide the advantage of helping to meet this need.

Prior to the present invention, there had been no demonstration or suggestion that translation control, and especially modulation of eIF-4E activity through 4E-BP1, could have an effect on glucose metabolism, fat tissue growth or weight gain. In view of the complexity of translation control, prior to the present invention, there was no teachings or suggestion that a knockout of 4E-BP1 could have such significant impact on the metabolism of an animal. In addition, prior to the present invention, there was no reasonable prediction that an inhibition of the interaction between 4E-BP1 and eIF-4E could modulate fat tissue growth, metabolism, glucose metabolism and weight gain in vivo. Indeed, in view of the stringent and complex regulation operating through eIF-4E on translation control in cells, and the demonstration that overexpression thereof could transform cells, and lead to tumors in animals (Sonenberg, 1996, mRNA 5′ Cap-binding Protein eIF4E and Control of Cell Growth. In Translational Control; eds. Hershey et al. 245-270, Cold Spring Harbor Laboratory Press, Cold Spring Harbor, N.Y.; and Lazaris-Karatzas et al., 1990, Nature 345:544-547), it could not be reasonably predicted that a knockout of 4E-BP1 could show such a significant and specific impact on the metabolism of the animal while the animal apparently remains physiologically normal. In addition, in view of the different eIF-4E binding proteins modulating the translation thereof, it could not be reasonably predicted that the knockout of 4E-BP1 would not be compensated by the other factors interacting with eIF-4E.

In a further general aspect, the invention relates to 4E-BP1 as a target to regulate fat tissue growth, metabolism, glucose metabolism, weight gain and energy homeostasis in vivo. 4E-BP1, cell lines and animals of the present invention can now be used to screen for regulators of 4E-BP1 activity and level, as well as 4E-BP1-eIF-4E interaction. The present invention thus provides the means to identify small diffusible ligands which can modulate the activity of the 4E-BP1 and of its interaction or sequestering of eIF-4E in vivo. Thus, the present invention also relates to agents or compounds that can desequester eIF-4E and/or affect the interaction between same and 4E-BP1. In essence therefore, the invention relates in part to agents which can modulate the interaction between inactive translational complexes and active ones (e.g. eIF-4F). It will be recognized by the person skilled in the art that having demonstrated the implication of cap-dependent translation in fat metabolism, glucose metabolism and weight gain, the present invention can be readily adapted to increase weight gain, fat tissue growth and the like. Thus,the present invention also relates to a sequestration of eIF-4E and to compounds and agents to promote same. Broadly therefore, the present invention relates to a method to modulate fat tissue growth, metabolism, glucose metabolism and weight gain in vivo and to methods to identify agents which affect such a modulation.

In addition, the invention relates to a method of producing a transgenic non-human animal displaying a lean phenotype the non-human mammal lacking expression of the endogenous 4E-BP1 polypeptide, the method including a disruption of the DNA encoding 4E-BP1, and a selection of progeny whose germ cells and somatic cells contain a knockout mutation in DNA encoding 4E-BP1, thereby yielding a lean non-human transgenic animal. Of course such lean transgenic animals could also be produced using a reduced amount of 4E-BP1 (e.g. using antisense 4E-BP1, for example), as opposed to a total abrogation of its expression or an antibody specific to 4E-BP1. In addition, animals expressing a nucleic acid sequence enabling an inhibition of the interaction between 4E-BP1 and eIF4E could also be produced. It should be understood that the present invention also provides methods of producing a fatter transgenic non-human animal, this transgenic animal having a level of sequestered eIF-4E which is higher than that of a control animal. In one particular embodiment, this sequestration is effected by an overexpression of 4E-BP1 or of a fragment or variant thereof which interacts with eIF-4E.

In a preferred embodiment, the invention relates to transgenic mice homozygous for the 4E-BP1 mutation, the mice being viable and fertile but exhibiting a significant reduction in adipose tissue content, glucose homeostasis and metabolic rate, as well as possible weight loss, while displaying apparently normal health.

Furthermore, the present invention relates to the demonstration that the 4E-BP1-eIF-4E interaction modulates fat tissue metabolism, glucose metabolism, metabolic rate and in some instances weight maintenance in an animal, thereby providing a new target for the development of therapeutics for obesity, fat deposition disorders and related diseases, as well as glucose metabolism-related diseases such as diabetes.

The present invention further relates to 4E-BP1-deficient non-human animals as a new model for the investigation of lipid metabolism, glucose metabolism, energy homeostasis and associated diseases.

In another aspect, the invention features a method of producing a transgenic non-human animal capable of expressing a functionally active non endogenous 4E-BP1 polypeptide, the non-human animal lacking expression of the endogenous 4E-BP1 polypeptide, the method including: (a) providing a transgenic non-human animal whose germ cells and somatic cells are deficient in 4E-BP1 (e.g. 4E-BP1 knockout); (b) introducing a non endogenous 4E-BP1 transgene capable of expressing a 4E-BP1 polypeptide, into a cell of the non-human animal; and (c) obtaining progeny expressing the non-endogenous transgene. In a preferred embodiment, the non endogenous 4E-BP1 transgene is a human transgene. In an especially preferred embodiment, the non endogenous transgene will be expressed in obesity- or diabetes-implicated cells and tissues.

Thus, the present invention also relates to a knock-in approach, by which a wild type or mutant copy of the 4E-BP1 gene (e.g. human) is introduced or replaces the disrupted copy of the endogenous 4E-BP1 gene. The knock-in approach has been described (Hanks et al., 1995, Science 269:679-682) and has been shown to enable the expression of the non-endogenous copy of the gene in the same cells as that of the endogenous gene.

In a related aspect, the present invention relates to the use of such non-human transgenic animals expressing a non-endogenous 4E-BP1 transgene to screen for a compound or agent that modulates 4E-BP1 activity, or 4E-BP1-eIF-4E interaction, the method including: exposing the non-human transgenic animal of the invention to the candidate compound, and determining the activity of the 4E-BP1 in the animal, wherein an increase in translation as compared to untreated non-human animals is indicative of a compound being capable of decreasing 4E-BP1 activity, or of decreasing the interaction or sequestration of eIF-4E by 4E-BP1, while an decrease in translation as compared to untreated non-human mammals is indicative of a compound being capable of increasing 4E-BP1 activity, or of increasing the interaction or sequestration of eIF-4E by 4E-BP1. In a preferred embodiment, the method further includes a determination of body or physiology parameters. Non-limiting examples thereof comprise a determination of at least one of: mass, body temperature, body fat content, fat to lean mass ratio, white adipose tissue deposits, white adipose tissue and/or brown adipose tissue, multilocular adipocyte, lipid droplet, expression level of UCP1 and/or UCP2, basal metabolic rate, food intake, hepatic synthetic functions, fasting serum triglyceride, serum glucose levels, level of expression of uncoupling protein mRNA in brown adipose tissue (BAT) and skeletal muscle, adipocyte volume in fat pads, lipogenesis, and fatty acid esterification and oxidation.

As it will be understood by the person of ordinary skill, the present invention may provide a number of significant advantages. For example, as for transgenic animals in general which have been shown to be useful for the investigation of biological processes and as animal model systems for general and specific aspects of health sciences in humans, the transgenic animals of the present invention provide a significant and pertinent model system for screening drugs to isolate therapeutic agents. In a particular embodiment, the novel transgenic animals of the present invention enable the selection and identification of modulators of the expression and/or activity of 4E-BP1. In a preferred embodiment, these agents have use as anti-obesity, anti-fat deposition disorders, anti-diabetes and anti-metabolic diseases associated with fat deposition disorders.

In one particular embodiment, the present invention relates to a method for identifying a compound having the ability to modulate energy homeostasis, glucose metabolism and/or lipid metabolism comprising: a) contacting this compound with a first peptide comprising an eIF-4E interaction domain and a second peptide comprising a sequence which directly interacts with this first peptide by direct binding, wherein a modulator of this direct binding is identified when same is significantly different in the presence of the compound as compared to in the absence thereof, and b) administering the compound selected as a modulator of this direct binding to an animal and measuring selected physiological and/or biochemical parameters, thereby enabling a determination as to whether this selected agent modulates glucose and/or fat metabolism in vivo.

It will also be apparent to the person of ordinary skill, to which this application pertains, that the transgenic animals of the present invention can further be bred with other animals harboring known genotypes associated with lipid metabolism-, glucose metabolism- or metabolism- related disorders. Similarly the transgenic mammals of the present invention can be used in biochemical experiments and the like designed to further understand, dissect and/or treat obesity and related disorders.

It will also be apparent that the cells and tissues of the transgenic animals of the present invention can be useful in in vitro methods relating to fat deposition and related disorders (including rational design and/or screening of compounds which can modulate expression and/or activity of the 4E-BP1). In a related aspect, the present invention further relates to cell lines in which the activity of 4E-BP1 (as it relates to its sequestration of eIF-4E) has been altered. In addition to being derived from the transgenic animals of the present invention, such cell lines, can for example be derived as commonly known in the art using the construct of the present invention or derivatives or variants thereof. Such cell lines can be used similarly to the animals of the present invention to identify compounds which modulate 4E-BP1 level and/or activity, dissect the physiological and biochemical function (including structure/function relationships, as they relate to translation and lipid and glucose metabolism) of 4E-BP1. Thus, the present invention also relates to established cell lines or primary cells derived from an animal of the present invention. As well, cell lines derived from 4E-BP1 knockout animal in accordance with the present invention can be transfected with a wild type or modified 4E-BP1 according to known methods. Such cell lines can be used in numerous assays and methods. Non-limiting examples of such assays are described in U.S. Pat. No. 5,874,231, or exemplified below.

Having determined that 4E-BP1, and eIF-4E are involved in lipid metabolism- and glucose metabolism-related disorders, as described herein, the present invention identifies 4E-BP1, eIF-4E and the interaction between 4E-BP1 and eIF-4E as targets for therapy and diagnosis of such disorders. Further, the present invention provides the means to modulate the activity/level of 4E-BP1. For example, antisense to 4E-BP1 can be used to decrease or abrogate the expression of 4E-BP1 polypeptide. This is expected to be associated with a lean phenotype. Antibodies, peptides, pharmaceutical ligands, small molecules and the like could be used with the same effect on the modulation of the interaction between 4E-BP1 and eIF-4E. Alternatively, in certain embodiments, the fat deposition could be increased by for example overexpressing 4E-BP1 in cells or tissues. Of course, the non-limiting agents mentioned above could also act as stimulators or agonists of this interaction.

Although the instant description focuses on mammalian transgenic animals, the present invention may also find utility in less common transgenic animals such as transgenic poultry. The production of leaner poultry might also be an advantage in the meat industry.

The invention therefore concerns transgenic animals, more particularly transgenic mammals and more specifically transgenic mice. In one particular embodiment of the present invention, the transgenic animal is a mice having both copies of the 4E-BP1 disrupted and hence no detectable 4E-BP1 protein.

The present invention further relates to the identification of eIF-4E sequestration and the modulation of 4E-BP1 as targets to modulate body metabolism in an animal.

Having now identified 4E-BP1 as a target for fat tissue growth modulation, glucose metabolism, fat modulation, diabetes, weight gain, energy homeostasis and the like, opens the way to the identification of further targets in the same pathway (i.e. translation control). Non-limiting examples of such targets include eIF4E, eIF-4F, kinases, phosphatases or other agents affecting the 4E-BP1 - eIF-4E interaction, or affecting the activity and/or the level of eIF-4E.

Further, the invention relates to methods of producing human transgenic animals and cell lines derived therefrom.

Also, the invention relates to assays and methods to identify agents which modulate glucose or fat metabolism, energy homeostasis and the like, by affecting the level and/or activity of eIF-4E or eIF-4F.

Prior to the present invention, the interaction between eIF-4E and 4E-BP1 and its role on animal physiology in vivo had not been assessed. The present invention broadly concerns the identification of translation as a critical biochemical process regulating fat tissue growth, metabolism, glucose metabolism and weight gain. More particularly, the invention identifies cap-dependent translation as a critical regulator of these processes. Even more particularly, the present invention pertains to the identification of 4E-BP1 as a regulator of these processes. Having demonstrated that a 4E-BP1 null mutation, (resulting in an increased availability of eIF-4E for eIF-4F formation), thereby enhancing cap-dependent translation, may play a role in obesity, it would be of interest to investigate the effects of leptin in 4E-BP1 knockout animals (e.g. knockout mice). Leptin is a hormone which is secreted by fat cells and works on the hypothalamus to depress appetite and other physiological response. For example, when fat tissue increases, a decrease in leptin amount is accompanied by an appetite increase. Of note, the knockout mouse of the present invention showed a 60% decrease in leptin. However, food intake was not significantly modified. Thus, these mice must regulate their food intake through a different mechanism. The knockout mice of the present invention could thus serve as an ideal model system to identify this mechanism of food intake regulation.

Analysis of brown fat in the knockout mice of the present invention showed that white fat tissue seems to be replaced by brown fat tissue, which contains an uncoupling protein generating heat by short-circuiting the mitochondrial proton battery. This could well explain the higher metabolic rate of the 4E-BP1 knockout mice. Once again, the 4E-BP1 knockout mice and cell lines derived therefrom could serve as ideal systems to test this hypothesis.

In a further general aspect of the present invention, there is also provided a method to modulate fat metabolism in cells and in animals comprising a sequestration or desequestration of eIF4E. In a related aspect, the method comprises a modulation of the level of eIF-4E in cells and tissue, thereby affecting fat metabolism.

For the purpose of the present invention, the following abbreviations and terms are defined below.

### DEFINITIONS

As used herein, the terminology “transgenic animal” refers to any animal which harbors a nucleic acid sequence having been inserted into a cell and having become part of the genome of the animal that develops from that cell. In a preferred embodiment, the transgenic animal is a mammal, in an especially preferred embodiment, the transgenic mammal is a mouse. However, other transgenic animals are encompassed as within scope of the present invention. Non-limiting examples of such transgenic animals include transgenic rodents (i.e. rats, hamsters, guinea pigs, and rabbits), and transgenic pigs, cattle and sheep, as well as transgenic poultry. Techniques for the preparation of such transgenic animals are well known in the art (e.g. introducing a transgene in ES cells; microinjecting the transgene into the male pronucleus of a fertilized egg; or infecting a cell with a recombinant virus). Indeed, lean transgenic animals find utility in the food industry, in view of the increasing awareness of consumers to the degree of fat in meat products. As used herein, “hon-human transgenic animal” is any non-human animal in which at least one cell comprises genetically altered information through known means such as microinjection, virus-delivered infection, or homologous recombination. In one particularly preferred embodiment of the present invention, the transgenic animal is a transgenic mouse, in which the genetic alteration has been introduced in a germ-line cell, such that it enables the transfer of this genetic alteration to the offsprings thereof. Such offsprings, containing this genetic alteration, are also transgenic mice.

The terminology “gene knockout” or “knockout” refers to a disruption of a nucleic acid sequence which significantly reduces and preferably suppresses or destroys the biological activity of the polypeptide encoded thereby. For example, 4E-BP1 knockout animal refers to an animal in which the expression of 4E-BP1 has been reduced or suppressed by the introduction of a recombinant nucleic acid molecule comprising 4E-BP1 sequences that disrupt at least a portion of the genomic DNA sequence encoding 4E-BP1 in the animal. A knockout animal might have one or both copies of the preselected nucleic acid sequence disrupted. In the latter case, in which a homozygous disruption is present, the mutation is termed a “null” mutation. In a case where only one copy of a preselected nucleic acid sequence is disrupted, the knockout animal is a “heterozygous knockout animal”.

The terminology “eIF-4E desequestering agent” refers to an agent which desequesters eIF-4E from an interaction with a cap-dependent translation inhibitor or down regulator (e.g. inhibited complex). More particularly, the terminology refers to an agent which interacts with eIF-4E or a sequestering agent thereof and alters the interaction thereof, in such a manner that it reduces or abrogates the sequestration of eIF-4E by the sequestering agent, thereby desequestering eIF-4E and increasing the translation of eIF-4E-dependent mRNAs. Non-limiting examples of eIF-4E sequestering agents include 4E-BP1, 4E-BP2, 4E-BP3, and fragments or variants thereof. Mutations in the coding sequence of eIF-4E sequestering agents and especially in the eIF-4E binding domain thereof are known in the art and further mutations could be readily obtained. It should be understood that eIF-4E sequestering agents have the opposite effect (i.e. they promote a decrease in the eIF-4E-dependent translation).

The term “fragment”, as applied herein to a peptide, refers to at least 7 contiguous amino acids, preferably about 14 to 16 contiguous amino acids, and more preferably, more than 40 contiguous amino acids in length. Such peptides can be produced by well-known methods to those skilled in the art, such as, for example, by proteolytic+ cleavage, genetic engineering or chemical synthesis.

The terminology “translation factor”, as commonly known in the art, is meant to refer to a group of factors or molecules participating directly in the translation of mRNA into polypeptides. Non-limiting examples thereof include eIF-1, eIF-2, eIF-3 and eIF-4A, eIF-4B, eIF-4E, eIF-4F, and eIF-4G.

The terminology “modulation of two factors” is meant to refer to a change in the affinity, strength, rate and the like between such two factors. The terminology “modulation of translation” refers to change in the efficiency or rate of translation of mRNAs resulting in a quantitative or qualitative change or rate of protein synthesis.

The terminology “eIF4E-dependent translation” is meant to refer to translation of an mRNA which requires eIF4E for its initiation of translation. As commonly known in the art, different mRNAs show different degrees of dependency on eIF4E for initiation of translation. The presence of the cap structure, consisting of a 7-methylguanosine residue linked to the 5′ position of eukaryotic mRNAs, and the degree of secondary structure between the cap structure and the initiator AUG, are two non-limiting factors which influence the dependency of an mRNA to eIF4E.

Nucleotide sequences are presented herein by single strand, in the 5′ to 3′ direction, from left to right, using the one letter nucleotide symbols as commonly used in the art and in accordance with the recommendations of the IUPAC-IUB Biochemical Nomenclature Commission.

Unless defined otherwise, the scientific and technological terms and nomenclature used herein have the same meaning as commonly understood by a person of ordinary skill to which this invention pertains. Generally, the procedures for cell cultures, infection, molecular biology methods and the like are common methods used in the art. Such standard techniques can be found in reference manuals such as for example Sambrook et al. (1989, Molecular Cloning - A Laboratory Manual, Cold Spring Harbor Laboratories) and Ausubel et al. (1994, Current Protocols in Molecular Biology, Wiley, N.Y.).

The present description refers to a number of routinely used recombinant DNA (rDNA) technology terms. Nevertheless, definitions of selected examples of such rDNA terms are provided for clarity and consistency.

As used herein, “nucleic acid molecule”, refers to a polymer of nucleotides. Non-limiting examples thereof include DNA (e.g. genomic DNA, cDNA) and RNA molecules (e.g. mRNA). The nucleic acid molecule can be obtained by cloning techniques or synthesized. DNA can be double-stranded or single-stranded (coding strand or non-coding strand [antisense]).

The term “recombinant DNA” as known in the art refers to a DNA molecule resulting from the joining of DNA segments. This is often referred to as genetic engineering.

The term “DNA segment”, is used herein, to refer to a DNA molecule comprising a linear stretch or sequence of nucleotides. This sequence when read in accordance with the genetic code, can encode a linear stretch or sequence of amino acids which can be referred to as a polypeptide, protein, protein fragment and the like.

The terminology “amplification pair” refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

The nucleic acid (e.g. DNA or RNA) for practising the present invention may be obtained according to well known methods.

Oligonucleotide probes or primers of the present invention may be of any suitable length, depending on the particular assay format and the particular needs and targeted genomes employed. In general, the oligonucleotide probes or primers are at least 12 nucleotides in length, preferably between 15 and 24 nucleotides, and they may be adapted to be especially suited to a chosen nucleic acid amplification system. As commonly known in the art, the oligonucleotide probes and primers can be designed by taking into consideration the melting point of hydrizidation thereof with its targeted sequence (see below and in Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

The term “oligonucleotide” or “DNA” molecule or sequence refers to a molecule comprised of the deoxyribonucleotides adenine (A), guanine (G), thymine (T) and/or cytosine (C), in a double-stranded form, and comprises or includes a “regulatory element” according to the present invention, as the term is defined herein. The term “oligonucleotide” or “DNA” can be found in linear DNA molecules or fragments, viruses, plasmids, vectors, chromosomes or synthetically derived DNA. As used herein, particular double-stranded DNA sequences may be described according to the normal convention of giving only the sequence in the 5′ to 3′ direction.

“Nucleic acid hybridization” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 1989, supra and Ausubel et al., 1989, supra) and are commonly known in the art. In the case of a hybridization to a nitrocellulose filter, as for example in the well known Southern blotting procedure, a nitrocellulose filter can be incubated overnight at 65° C. with a labelled probe in a solution containing 50% formamide, high salt (5×SSC or 5×SSPE), 5×Denhardt's solution,1% SDS, and 100/μg/ml denatured carrier DNA (e.g. salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The selected temperature is based on the melting temperature (Tm) of the DNA hybrid. Of course, RNA-DNA hybrids can also be formed and detected. In such cases, the conditions of hybridization and washing can be adapted according to well known methods by the person of ordinary skill. Stringent conditions will be preferably used (Sambrook et al.,1989, supra).

Probes of the invention can be utilized with naturally occurring sugar-phosphate backbones as well as modified backbones including phosphorothioates, dithionates, alkyl phosphonates and a-nucleotides and the like. Modified sugar-phosphate backbones are generally taught by Miller, 1988, Ann. Reports Med. Chem. 23:295 and Moran et al., 1987, Nucleic acid molecule. Acids Res., 14:5019. Probes of the invention can be constructed of either ribonucleic acid (RNA) or deoxyribonucleic acid (DNA), and preferably of DNA.

The types of detection methods in which probes can be used include Southern blots (DNA detection), dot or slot blots (DNA, RNA), and Northern blots (RNA detection). Although less preferred, labelled proteins could also be used to detect a particular nucleic acid sequence to which it binds. Other detection methods include kits containing probes on a dipstick setup and the like.

Although the present invention is not specifically dependent on the use of a label for the detection of a particular nucleic acid sequence, such a label might be beneficial, by increasing the sensitivity of the detection. Furthermore, it enables automation. Probes can be labelled according to numerous well known methods (Sambrook et al., 1989, supra). Non-limiting examples of labels include ³H, ¹⁴C, ³²P, and 35S. Non-limiting examples of detectable markers include ligands, fluorophores, chemiluminescent agents, enzymes, and antibodies. Other detectable markers for use with probes, which can enable an increase in sensitivity of the method of the invention, include biotin and radionucleotides. It will become evident to the person of ordinary skill that the choice of a particular label dictates the manner in which it is bound to the probe.

As commonly known, radioactive nucleotides can be incorporated into probes of the invention by several methods. Non-limiting examples thereof include kinasing the 5′ ends of the probes using gamma ³²P ATP and polynucleotide kinase, using the Klenow fragment of Pol I of E. coli in the presence of radioactive dNTP (e.g. uniformly labelled DNA probe using random oligonucleotide primers in low-melt gels), using the SP6/T7 system to transcribe a DNA segment in the presence of one or more radioactive NTP, and the like.

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill. An oligonucleotide can be synthetised chemically or derived by cloning according to well known methods.

As used herein, a “primer” defines an oligonucleotide which is capable of annealing to a target sequence, thereby creating a double stranded region which can serve as an initiation point for DNA synthesis under suitable conditions.

Amplification of a selected, or target, nucleic acid sequence may be carried out by a number of suitable methods. See generally Kwoh et al., 1990, Am. Biotechnol. Lab. 8:14-25. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the Qβ replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra). Preferably, amplification will be carried out using PCR.

Polymerase chain reaction (PCR) is carried out in accordance with known techniques. See, e.g., U.S. Pat. Nos. 4,683,195; 4,683,202; 4,800,159; and 4,965,188 (the disclosures of all three U.S. Patent are incorporated herein by reference). In general, PCR involves, a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analysed to assess whether the sequence or sequences to be detected are present. Detection of the amplified sequence may be carried out by visualization following EtBr staining of the DNA following gel electrophores, or using a detectable label in accordance with known techniques, and the like. For a review on PCR techniques (see PCR Protocols, A Guide to Methods and Amplifications, Michael et al. Eds, Acad. Press, 1990).

Ligase chain reaction (LCR) is carried out in accordance with known techniques (Weiss, 1991, Science 254:1292). Adaptation of the protocol to meet the desired needs can be carried out by a person of ordinary skill. Strand displacement amplification (SDA) is also carried out in accordance with known techniques or adaptations thereof to meet the particular needs (Walker et al., 1992, Proc. NatI. Acad. Sci. USA 89:392-396; and ibid., 1992, Nucleic Acids Res. 20:1691-1696).

As used herein, the term “gene” is well known in the art and relates to a nucleic acid sequence defining a single protein or polypeptide. A “structural gene” defines a DNA sequence which is transcribed into RNA and translated into a protein having a specific amino acid sequence thereby giving rise to a specific polypeptide or protein. It will be readily recognized by the person of ordinary skill, that the nucleic acid sequence of the present invention can be incorporated into anyone of numerous established kit formats which are well known in the art.

A “heterologous” (e.g. a heterologous gene) region of a DNA molecule is a subsegment segment of DNA within a larger segment that is not found in association therewith in nature. The term “heterologous” can be similarly used to define two polypeptidic segments not joined together in nature. Non-limiting examples of heterologous genes include reporter genes such as luciferase, chloramphenicol acetyl transferase, β-galactosidase, and the like which can be juxtaposed or joined to heterologous control regions or to heterologous polypeptides.

The term “vector” is commonly known in the art and defines a plasmid DNA, phage DNA, viral DNA and the like, which can serve as a DNA vehicle into which DNA of the present invention can be cloned. Numerous types of vectors exist and are well known in the art.

The term “expression” defines the process by which a gene is transcribed into mRNA (transcription), the mRNA is then being translated (translation) into one polypeptide (or protein) or more.

The terminology “expression vector” defines a vector or vehicle as described above but designed to enable the expression of an inserted sequence following transformation into a host. The cloned gene (inserted sequence) is usually placed under the control of control element sequences such as promoter sequences. The placing of a cloned gene under such control sequences is often referred to as being operably linked to control elements or sequences.

Operably linked sequences may also include two segments that are transcribed onto the same RNA transcript. Thus, two sequences, such as a promoter and a “reporter sequence” are operably linked if transcription commencing in the promoter will produce an RNA transcript of the reporter sequence. In order to be “operably linked” it is not necessary that two sequences be immediately adjacent to one another.

Expression control sequences will vary depending on whether the vector is designed to express the operably linked gene in a prokaryotic or eukaryotic host or both (shuttle vectors) and can additionally contain transcriptional elements such as enhancer elements, termination sequences, tissue-specificity elements, and/or translational initiation and termination sites.

Prokaryotic expressions are useful for the preparation of large quantities of the protein encoded by the DNA sequence of interest. This protein can be purified according to standard protocols that take advantage of the intrinsic properties thereof, such as size and charge (e.g. SDS gel electrophoresis, gel filtration, centrifugation, ion exchange chromatography...). In addition, the protein of interest can be purified via affinity chromatography using polyclonal or monoclonal antibodies. The purified protein can be used for therapeutic applications.

The DNA construct can be a vector comprising a promoter that is operably linked to an oligonucleotide sequence of the present invention, which is in turn, operably linked to a heterologous gene, such as the gene for the luciferase reporter molecule. “Promoter” refers to a DNA regulatory region capable of binding directly or indirectly to RNA polymerase in a cell and initiating transcription of a downstream (3′ direction) coding sequence. For purposes of the present invention, the promoter is bound at its 3′ terminus by the transcription initiation site and extends upstream (5′ direction) to include the minimum number of bases or elements necessary to initiate transcription at levels detectable above background. Within the promoter will be found a transcription initiation site (conveniently defined by mapping with S1 nuclease), as well as protein binding domains (consensus sequences) responsible for the binding of RNA polymerase. Eukaryotic promoters will often, but not always, contain “TATA” boses and “CCAT” boxes. Prokaryotic promoters contain Shine-Dalgarno sequences in addition to the −10 and −35 consensus sequences.

As used herein, the designation “functional derivative” denotes, in the context of a functional derivative of a sequence whether an nucleic acid or amino acid sequence, a molecule that retains a biological activity (either function or structural) that is substantially similar to that of the original sequence. This functional derivative or equivalent may be a natural derivative or may be prepared synthetically. Such derivatives include amino acid sequences having substitutions, deletions, or additions of one or more amino acids, provided that the biological activity of the protein is conserved. The same applies to derivatives of nucleic acid sequences which can have substitutions, deletions, or additions of one or more nucleotides, provided that the biological activity of the sequence is generally maintained. When relating to a protein sequence, the substituting amino acid as chemico-physical properties which are similar to that of the substituted amino acid. The similar chemico-physical properties include, similarities in charge, bulkiness, hydrophobicity, hydrophylicity and the like. The term “functional derivatives” is intended to include “fragments”, “segments”, “variants”, “analogs” or “chemical derivatives” of the subject matter of the present invention.

As well-known in the art, a conservative mutation or substitution of an amino acid refers to mutation or substitution which maintains 1) the structure of the backbone of the polypeptide (e.g. a beta sheet or alpha-helical structure); 2) the charge or hydrophobicity of the amino acid; or 3) the bulkiness of the side chain. More specifically, the well-known terminologies “hydrophilic residues” relate to serine or threonine. “Hydrophobic residues” refer to leucine, isoleucine, phenylalanine, valine or alanine. “Positively charged residues” relate to lysine, arginine or hystidine. Negatively charged residues” refer to aspartic acid or glutamic acid. Residues having “bulky side chains” refer to phenylalanine, tryptophan or tyrosine.

Peptides, protein fragments, and the like in accordance with the present invention can be modified in accordance with well-known methods dependently or independently of the sequence thereof. For example, peptides can be derived from the wild-type sequence exemplified herein in the figures using conservative amino acid substitutions at 1, 2, 3 or more positions. The terminology “conservative amino acid substitutions” is well-known in the art which relates to substitution of a particular amino acid by one having a similar characteristic (e.g. aspartic acid for glutamic acid, or isoleucine for leucine). Of course, non-conservative amino acid substitutions can also be carried out, as well as other types of modifications such as deletions or insertions, provided that these modifications modify the peptide, in a suitable way (e.g. without affecting the biological activity of the peptide if this is what is intended by the modification). A list of exemplary conservative amino acid substitutions is given hereinbelow.

TABLE 2
CONSERVATIVE AMINO ACID REPLACEMENTS

| For Amino Acid | Code | Replace With |
|---|---|---|
| Alanine | A | D-Ala, Gly, Aib, ∃-Ala, Acp, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, Aib, ∃-Ala, Acp |
| Isoleucine | I | D-Ile, Val, D-Val, AdaA, AdaG, Leu, D-Leu, |
| | | Met, D-Met |
| Leucine | L | D-Leu, Val, D-Val, AdaA, AdaG, Leu, D-Leu. |
| | | Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, |
| | | Val, D-Val |
| Phenylalanine | F | D-Phe, Tyr, D-Thr, L-Dopa, His, D-His, |
| | | Trp, D-Trp, Trans-3,4, or 5-phenylproline, |
| | | AdaA, AdaG, cis-3,4, or 5-phenylproline, |
| | | Bpa, D-Bpa |
| Proline | P | D-Pro, L-I-thioazolidine-4-carboxylic |
| | | acid, D-or L-1-oxazolidine-4-carboxylic |
| | | acid (Kauer, U.S. Pat. No. (4,511,390) |
| Serine | S | D-Ser, Thr, D-Thr, allo-Thr, Met, D-Met, |
| | | Met (O), D-Met(O), L-Cys, D-Cys |
| Threonine | T | D-Thr, Ser, D-Ser, allo-Thr, Met, D-Met, |
| | | Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met, |
| | | AdaA, AdaG |

As can be seen in this table, some of these modifications can be used to render the peptide more resistant to proteolysis. Of course, modifications of the peptides can also be effected without affecting the primary sequence thereof using enzymatic or chemical treatment as well-known in the art.

Thus, the term “variant” refers herein to a protein or nucleic acid molecule which is substantially similar in structure and biological activity to the protein or nucleic acid of the present invention.

Although exemplified with a 4E-BP1 knockout leading to desequesterization of eIF-4E, it should be clear to the skilled artisan that the present invention should not be so limited. For decreasing adipose tissue for example, a number of means to desequester eIF4E are available. Non-limiting examples include knockouts (or a decrease in the level or activity of an eIF-4E sequestering agent as explained above) or mutations in 4E-BP2 and 4E-BP3. Conversely, to increase adipose tissue, increase weight gain or the like, a number of eIF-4E sequestering agents could be used. Non-limiting examples thereof of proteins or fragments thereof which could be used to sequester eIF4E include proteins or amino acid sequences comprising an eIF4E binding site. Examples of such proteins include 4E-BP1, 4E-BP2, 4E-BP3 and eIF-4G. In addition, in view of the conservation of the eIF4E binding domain of such proteins during evolution, numerous sequences can be synthesized or derived from diverse animal and plant sources.

The functional derivatives of the present invention can be synthesized chemically or produced through recombinant DNA technology, all these methods are well known in the art. In one particular embodiment of the present invention, a variant according to the present invention includes an eIF4E sequestering agent, such as a 4E-BP1 variant or fragment which retains its ability in sequestering eIF4E, thereby modulating translation initiation and consequently the fat and glucose metabolism. The interaction domains of eIF4E and 4E-BP1 being known, it is thus possible for the skilled artisan to identify and/or design 4E-BP1 variants having a modified affinity for eIF4E (see the alignments below). In addition, having identified eIF4E-dependent translation as a key biochemical process involved in glucose and fat metabolism in vivo, the present invention provides the means to influence these processes by modifying the domain of eIF4E which interacts with different sequestering agents, or using agents which target eIF4E or other factors with which it interacts so that a modulation of eIF4E interactions with different initiation factors can occur.

As used herein, “chemical derivatives” is meant to cover additional chemical moieties not normally part of the subject matter of the invention. Such moieties could affect the physico-chemical characteristic of the derivative (e.g. solubility, absorption, half life and the like, decrease of toxicity). Such moieties are exemplified in Remington's Pharmaceutical Sciences (e.g. 1980). Methods of coupling these chemical-physical moieties to a polypeptide are well known in the art.

The term “allele” defines an alternative form of a gene which occupies a given locus on a chromosome.

As commonly known, a “mutation” is a detectable change in the genetic material which can be transmitted to a daughter cell. As well known, a mutation can be, for example, a detectable change in one or more deoxyribonucleotide. For example, nucleotides can be added, deleted, substituted for, inverted, or transposed to a new position. Spontaneous mutations and experimentally induced mutations exist. The result of a mutations of nucleic acid molecule is a mutant nucleic acid molecule. A mutant polypeptide can be encoded from this mutant nucleic acid molecule.

As used herein, the term “purified” refers to a molecule having been separated from a cellular component. Thus, for example, a “purified protein” has been purified to a level not found in nature. A “substantially pure” molecule is a molecule that is lacking in most other cellular components.

As used herein, the terms “molecule”, “compound” or “ligand” are used interchangeably and broadly to refer to natural, synthetic or semi-synthetic molecules or compounds. The term “molecule” therefore denotes for example chemicals, macromolecules, cell or tissue extracts (from plants or animals) and the like. Non limiting examples of molecules include nucleic acid molecules, peptides, antibodies, carbohydrates and pharmaceutical agents. The agents can be selected and screened by a variety of means including random screening, rational selection and by rational design using for example protein or ligand modelling methods such as computer modelling, combinatorial library screening and the like. The terms “rationally selected” or “rationally designed” are meant to define compounds which have been chosen based on the configuration of the interaction domains of the present invention. As will be understood by the person of ordinary skill, macromolecules having non-naturally occurring modifications are also within the scope of the term “molecule”. For example, peptidomimetics, well known in the pharmaceutical industry and generally referred to as peptide analogs can be generated by modelling as mentioned above. Similarly, in a preferred embodiment, the polypeptides of the present invention are modified to enhance their stability. It should be understood that in most cases this modification should not alter the biological activity of the interaction domain. The molecules identified in accordance with the teachings of the present invention have a therapeutic value in diseases or conditions in which the physiology or homeostasis of the cell and/or tissue is compromised by a defect in fat tissue metabolism and/or glucose metabolism, and/or obesity, and/or diabetes. Alternatively, the molecules identified in accordance with the teachings of the present invention find utility in the development of more efficient agents which can decrease or reverse a defect in fat tissue metabolism and/or glucose metabolism, and/or obesity, and/or diabetes.

Libraries of compounds (publicly available or commercially available) are well-known in the art. The term “compounds” is also meant to cover ribozymes (see, for example, U.S. Pat. No. 5,712,384, U.S. Pat. No. 5,879,938; and U.S. Pat. No. 4,987,071), and aptamers (see, for example, U.S. Pat No. 5,756,291 and U.S. Pat. No. 5,792,613).

In one particular embodiment of the present invention, peptides are used as agents to interfere with the eIF-4E-4E-BP1 interaction or to interfere with desequestration of eIF-4E. In one particular embodiment, a peptide capable of sequestering eIF-4E contains at least 7 amino acids, and preferably between at least 14 and 16 amino acids with at least 80% sequence identity to the amino acid sequences of the 4E-BPs shown below or of the 4E-binding sites also shown hereinbelow. In one particular embodiment of the present invention, the sequestering peptide comprises a consensus sequence selected from: YxxxxLφ, +φxxYx+xfφφ, +φφY−+xF/AφφxxRxSP, and +φφY−+xfLφxxRxSP. Preferably, the sequestering peptide having the ability to bind to eIF-4E and thereby modulate energy homeostasis in an animal contains between 7 and 16 amino acids with at least 95% sequence identity to the amino acid sequence of the 4E-binding sites shown in the figures below. More preferably, the sequestering peptide has a 100% sequence identity to the amino acid sequences shown in the figures and even more preferably, 100% sequence identity with mammalian 4E-binding sites and particularly human rat mouse 4E-binding sites. Conversely, it shall be understood that eIF-4E desequestering agents can in certain embodiments be selected from peptides which bind to 4E-BPs. In one particular embodiment, such peptides are selected from 4E-BP interaction domain of eIF-4E.

As used herein, agonists and antagonists of the eIF-4E-4E-BP1 interaction also include potentiators of known compounds with such agonist or antagonist properties. In one embodiment, agonists can be detected by contacting the indicator cell with a compound or mixture or library of molecules (e.g. a combinatorial library) for a fixed period of time and, for example, the translation activity is then determined.

In one embodiment, the level of gene expression of the reporter gene (e.g. the level of luciferase, or,β-gal, produced) within the treated cells can be compared to that of the reporter gene in the absence of the molecules(s). The difference between the levels of gene expression indicates whether the molecule(s) of interest agonizes the aforementioned interaction. The magnitude of the level of reporter gene product expressed (treated vs. untreated cells) provides a relative indication of the strength of that molecule(s) as an agonist. The same type of approach can also be used in the presence of an antagonist(s).

The present invention also provides antisense nucleic acid molecules which can be used for example to decrease or abrogate the expression of the nucleic acid sequences or proteins of the present invention. An antisense nucleic acid molecule according to the present invention refers to a molecule capable of forming a stable duplex or triplex with a portion of its targeted nucleic acid sequence (DNA or RNA). In one particular embodiment, the antisense is specific to 4E-BP1. The use of antisense nucleic acid molecules and the design and modification of such molecules is well known in the art as described for example in WO 96/32966, WO 96/11266, WO 94/15646, WO 93/08845 and U.S. Pat. No. 5,593,974. Antisense nucleic acid molecules according to the present invention can be derived from the nucleic acid sequences and modified in accordance to well known methods. For example, some antisense molecules can be designed to be more resistant to degradation to increase their affinity to their targeted sequence, to affect their transport to chosen cell types or cell compartments, and/or to enhance their lipid solubility bu using nucleotide analogs and/or substituting chosen chemical fragments thereof, as commonly known in the art.

Alternatively, an indicator cell in accordance with the present invention can be used to identify antagonists. For example, the test molecule or molecules are incubated with the host cell in conjunction with one or more agonists held at a fixed concentration. An indication and relative strength of the antagonistic properties of the molecule(s) can be provided by comparing the level of gene expression in the indicator cell in the presence of the agonist, in the absence of test molecules versus in the presence thereof. Of course, the antagonistic effect of a molecule could also be determined in the absence of agonist, simply by comparing the level of expression of the reporter gene product in the presence and absence of the test molecule(s).

It shall be understood that the “in vivo” experimental model can also be used to carry out an “in vitro” assay. For example, cellular extracts from the indicator cells and/or cellular extracts from the non-human transgenic animals of the present invention can be prepared and used in one of the in vitro method of the present invention or an in vitro method known in the art. Non-limiting examples of assays are exemplified herein and taught in U.S. Pat. No. 5,874,231.

As used herein the recitation “indicator cells” refers to cells that express, in one particular embodiment, the 4E-BP1 and eIF-4E or domains thereof which interact, and wherein an interaction between these proteins or interacting domains thereof is coupled to an identifiable or selectable phenotype or characteristic such that it provides an assessment of the interaction between same. Such indicator cells can be used in the screening assays of the present invention. In certain embodiments, the indicator cells have been engineered so as to express a chosen derivative, fragment, homolog, or mutant of these interacting domains. The cells can be yeast cells or higher eukaryotic cells such as mammalian cells (WO 96/41169). In one particular embodiment, the indicator cell is a yeast cell harboring vectors enabling the use of the two hybrid system technology, as well known in the art (Ausubel et al., 1994, supra) and can be used to test a compound or a library thereof. In one embodiment, a reporter gene encoding a selectable marker or an assayable protein can be operably linked to a control element such that expression of the selectable marker or assayable protein is dependent on the interaction of the eIF-4E and 4E-BP1 interacting domains. Such an indicator cell could be used to rapidly screen at high-throughput a vast array of test molecules. In a particular embodiment, the reporter gene is luciferase or β-Gal.

In one embodiment, at least one of the 4E-BP1 and eIF-4E interacting domains of the present invention may be provided as a fusion protein. The design of constructs therefor and the expression and production of fusion proteins are well known in the art (Sambrook et al., 1989, supra; and Ausubel et al., 1994, supra). In a particular embodiment, both interaction domains are part of fusion proteins. A non-limiting example of such fusion proteins includes a LexA-4E-BP1 fusion (DNA-binding domain-4E-BP1; bait) and a B42-eIF-4E fusion (transactivator domain-eIF-4E; prey). In yet another particular embodiment, the LexA-4E-BP1 and B42-eIF-4E fusion proteins are expressed in a yeast cell also harboring a reporter gene operably linked to a LexA operator and/or LexA responsive element. Of course, it will be recognized that other fusion proteins can be used in such 2 hybrid systems. Furthermore, it will be recognized that the fusion proteins need not contain the full-length 4E-BP1 or eIF-4E polypeptide. Indeed, fragments of these polypeptides, provided that they comprise the interacting domains, can be used in accordance with the present invention.

Non-limiting examples of such fusion proteins include a hemaglutinin fusions, Gluthione-S-transferase (GST) fusions and Maltose binding protein (MBP) fusions. In certain embodiments, it might be beneficial to introduce a protease cleavage site between the two polypeptide sequences which have been fused. Such protease cleavage sites between two heterologously fused polypeptides are well known in the art.

In certain embodiments, it might also be beneficial to fuse the interaction domains of the present invention to signal peptide sequences enabling a secretion of the fusion protein from the host cell. Signal peptides from diverse organisms are well known in the art. Bacterial OmpA and yeast Suc2 are two non limiting examples of proteins containing signal sequences. In certain embodiments, it might also be beneficial to introduce a linker (commonly known) between the interaction domain and the heterologous polypeptide portion. Such fusion protein find utility in the assays of the present invention as well as for purification purposes, detection purposes and the like.

For certainty, the sequences and polypeptides useful to practice the invention include without being limited thereto mutants, homologs, subtypes, alleles and the like. It shall be understood that generally, the sequences of the present invention should encode a functional (albeit defective) interaction domain. It will be clear to the person of ordinary skill that whether an interaction domain of the present invention, variant, derivative, or fragment thereof retains its function in binding to its partner can be readily determined by using the teachings and assays of the present invention and the general teachings of the art.

As exemplified herein below, the interaction domains of the present invention can be modified, for example by in vitro mutagenesis, to dissect the structure-function relationship thereof and permit a better design and identification of modulating compounds. However, some derivative or analogs having lost their biological function of interacting with their respective interaction partner (4E-BP1 or eIF-4E) may still find utility, for example for raising antibodies. Such analogs or derivatives could be used for example to raise antibodies to the interaction domains of the present invention. These antibodies could be used for detection or purification purposes. In addition, these antibodies could also act as competitive or non-competitive inhibitor and be found to be modulators of 4E-BP1-eIF-4E interaction.

A host cell or indicator cell has been “transfected” by exogenous or heterologous DNA (e.g. a DNA construct) when such DNA has been introduced inside the cell. The transfecting DNA may or may not be integrated (covalently linked) into chromosomal DNA making up the genome of the cell. In prokaryotes, yeast, and mammalian cells for example, the transfecting DNA may be maintained on a episomal element such as a plasmid. With respect to eukaryotic cells, a stably transfected cell is one in which the transfecting DNA has become integrated into a chromosome so that it is inherited by daughter cells through chromosome replication. This stability is demonstrated by the ability of the eukaryotic cell to establish cell lines or clones comprised of a population of daughter cells containing the transfecting DNA. Transfection methods are well known in the art (Sambrook et al., 1989, supra; Ausubel et al., 1994 supra). The use of a mammalian cell as indicator can provide the advantage of furnishing an intermediate factor, which permits for example the interaction of two polypeptides which are tested, that might not be present in lower eukaryotes or prokaryotes. Of course, an advantage might be rendered moot if both polypeptide tested directly interact. It will be understood that extracts from mammalian cells for example could be used in certain embodiments, to compensate for the lack of certain factors in a chosen indicator cell. It shall be realized that the field of translation provides ample teachings of methods to prepare and reconstitute translation extracts.

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody—A Laboratory Manual, CSH Laboratories). The present invention also provides polyclonal, monoclonal antibodies, or humanized versions thereof, chimeric antibodies and the like which inhibit or neutralize their respective interaction domains and/or are specific thereto.

From the specification and appended claims, the term therapeutic agent should be taken in a broad sense so as to also include a combination of at least two such therapeutic agents. Further, the DNA segments or proteins according to the present invention can be introduced into individuals in a number of ways. For example, erythropoietic cells can be isolated from the afflicted individual, transformed with a DNA construct according to the invention and reintroduced to the afflicted individual in a number of ways, including intravenous injection. Alternatively, the DNA construct can be administered directly to the afflicted individual, for example, by injection in the bone marrow. The DNA construct can also be delivered through a vehicle such as a liposome, which can be designed to be targeted to a specific cell type, and engineered to be administered through different routes.

In one particular embodiment, the present invention provides the means to treat weight-related diseases or conditions comprising a decrease or total eradication of 4E-BP1 expression. It will be recognized that having shown that the absence of 4E-BP1 expression reduces fat tissue, provides numerous means of achieving fat reduction in animals. Non-limiting examples of such means include 4E-BP1 anthozoans, 4E-BP1 ligands (e.g. antibodies), 4E-BP1 mutants (e.g. mutants in the eIF-4E interacting domain) and the like.

For administration to humans, the prescribing medical professional will ultimately determine the appropriate form and dosage for a given patient, and this can be expected to vary according to the chosen therapeutic regimen (e.g. DNA construct, protein, molecule), the response and condition of the patient as well as the severity of the disease.

Composition within the scope of the present invention should contain the active agent (e.g. protein, nucleic acid, or molecule) in an amount effective to achieve the desired therapeutic effect while avoiding adverse side effects. Typically, the nucleic acids in accordance with the present invention can be administered to mammals (e.g. humans) in doses ranging from 0.005 to 1 mg per kg of body weight per day of the mammal which is treated. Pharmaceutically acceptable preparations and salts of the active agent are within the scope of the present invention and are well known in the art (Remington's Pharmaceutical Science, 16th Ed., Mack Ed.). For the administration of polypeptides, antagonists, agonists and the like, the amount administered should be chosen so as to avoid adverse side effects. The dosage will be adapted by the clinician in accordance with conventional factors such as the extent of the disease and different parameters from the patient. Typically, 0.001 to 50 mg/kg/day will be administered to the mammal.

### BRIEF DESCRIPTION OF THE DRAWINGS

Having thus generally described the invention, reference will now be made to the accompanying drawings, showing by way of illustration a preferred embodiment thereof, and in which:

FIG. 1 shows the gene targeting of mouse 4E-BP1, a shows the restriction map of the targeting vector, mouse 4E-BP1 gene, and the structure of the mutated locus following homologous recombination, the coding exons are depicted by black boxes (2 and 3) and the open boxes denote the 3′ non-coding portion in the third exon, genomic fragments used as probes for southern blotting are shown by closed boxes (probe a, probe b and probe c), PCR region for genotyping is depicted as a black line (abbreviations and symbols: Neo, Neomycin transferase gene, B, BamHI, X, XbaI), b shows the southern blot analysis of genomic DNA from ES cell clones, the DNA was digested with Xbal and BamHl, and hybridized with probes a, b, and c, the sizes of wild type (WT) and disrupted (KO) alleles are shown; the genotypes of the ES cell clones are presented above the lanes and c illustrates the analysis of mouse progenies by genomic southern blotting, PCR and Western blotting, the sizes of WT. and KO alleles (Southern, probe b, BamHI), and that of the PCR amplification products are shown, the mice genotypes are indicated above the lanes;

FIG. 2 shows the mice glycaemia and insulin test. a illustrates that the blood glucose concentration of five fed male mice was determined (16:30) and b illustrates that five male mice were fasted for 6 h (3:00-9:00 AM), and insulin (Eli-Lilly) was injected (0.4 U/kg) intraperitoneally, blood was collected serially from retro-orbital sinus or tail vein under anesthesia and blood glucose levels were measured; the insulin tolerance test was performed twice and the mean and standard deviation from the mean are shown;

FIG. 3 shows that brown adipocytes are induced in Eif4ebp1^{-/-} mice. a, Sections of interscapular brown adipose tissue (IBAT) and inguinal and retroperitoneal white adipose tissue (IWAT and RWAT) from a wild-type (+/+) and an Eif4ebp1^{-/-} (−/−) male littermates. Sections were stained with hematoxylin and eosin. b, mRNA expression levels of the uncoupling protein 1 (UCP1), uncoupling protein 2 (UCP2) and actin in inguinal white adipose tissue depots from wild-type (+/+) and Eif4ebp1^{-/-} (−-−) male mice. Data were quantitated using a Phosphorlmager®) (Fuji). c, Quantitation of UCP1 and UCP2 mRNA expression. Levels were normalized to actin and are presented as mean ± s.e.m. Statistical analysis was performed using a Mann-Whitney test, and UCP1 levels were found to be significantly different (p<0.05) between wild-type and Eif4ebp1^{-/-} mice;

FIG. 4 shows that cap-dependent translation is increased in Eif4ebp1 (-/-) MEFs. a illustrates the structure of the expression vectors, T7-CAT and T7-EMCV-CAT: T7 transcription promoter; CAT: chloramphenicol acetyl transferase; EMCV: encephalomyocarditis virus; IRES: internal ribosomal entry site. b shows CAT protein synthesis in Eif4ebp1^{-/-} (−/−) and wild-type (+/+) MEFs. The monocistronic T7-CAT (shaded bar) and T7-EMCV-CAT RNA (hatched bar) were synthesized by T7 recombinant vaccinia virus expressing the T7 RNA polymerase (Yasui et al., 1998). Expressed CAT protein in Eif4ebp1^{+/+}and Eif4ebp1^{-/-} MEF cells are indicated, quantities of synthetic RNA in cells were measured by RTD-PCR (real time detection PCR). Data are presented as mean ± s.e.m. (standard error of the mean) of two experiments performed in triplicate. c shows the growth properties of mouse embryo fibroblasts. MEFs were prepared from 14 days embryos, as described. MEFs were maintained in DMEM containing 10% fetal calf serum and 10 mM HEPES, growth curves were determined for MEFs from passage 5 to 6, the genotype of each cell is indicated in the figure. Cells (10⁵) were plated in triplicate in 35 mm diameter dishes, counted in three independent experiments and the mean and standard deviation from the mean were calculated;

FIG. 5 shows that eIF4E phosphorylation is increased in Eif4ebp1^{-/-} (−/−) MEFs, a shows 4E-BP1 in Eif4ebp1^{+/+}(wild-type (+/+)) and Eif4ebp1^{-/-} MEFs. Cells were transfected with vector alone (vec.) or 4E-BP1 (BP1) and protein expression was detected by Western blot as described below. b shows the Phosphorylation state of eIF4E. Phosphorylation was assessed by isoelectricfocusing followed by Western blotting using a polyclonal anti-eIF-4E antibody, the intensity of the phosphorylated (P.I.=5.9) and de-phosphorylated (P.I.=6.3) eIF4E forms was measured by densitometry. Quantification of eIF4E illustrates phosphorylation. Data are presented as mean ± s.e.m. of three experiments;

FIG. 6 shows the sequence alignment of the 4E-binding site of 4E-BPs, as well as the consensus sequence which could be used as a 4E sequestering agent or for the development of further 4E sequestering agents. The light gray indicates positions at which mutation to alanine abrogates the binding to eIF-4E (Mader et al., 1995; and Poulin et al., 1998). The dark gray indicates highly conserved amino acid positions. +/− indicate charged amino acids. φ refers to hydrophobic amino acids, Y refers to tyrosine, f refers to phenylalanine, although an absolute requirement for this amino acid does not appear to be necessary based on the dyctostelium discoideum consensus sequence. L refers to leucine, and “.” shows that the 4E binding site at this particular position is not dependent on a particular amino acid. h=human (of note, mouse and rat have the same sequence in this region); gg=gallus gallus (chicken); hr=halocynthia roretzi; bm=bombyx mori; sm=schistosoma mansoni; dd=dictyostelium discoideum. Y, L, R, S, and P refer to the standard one letter code for amino acids; and

FIG. 7 shows the alignment of 4E-binding sites comprised in a number of diverse eIF4E-binding proteins. The light gray indicates positions at which a mutation to alanine abrogates the binding to eIF4E (Mader et al., 1995; and Poulin et al., 1998). +/− indicate charged amino acids. φ refers to hydrophobic amino acids. Y and L refer to the standard one letter code for amino acids.

Other objects, advantages and features of the present invention will become more apparent upon reading of the following non-restrictive description of preferred embodiments with reference to the accompanying drawing which is exemplary and should not be interpreted as limiting the scope of the present invention.

### DESCRIPTION OF THE PREFERRED EMBODIMENT

A method of production and the transgenic animals of the present invention is described herein below. In general, these animals are produced by engineering a nucleic acid construct which can disrupt the expression of the endogenous targeted gene (e.g. 4E-BP1 gene, and more particularly the murine 4E-BP1 gene). Using known methods, this construct is amplified in bacterial cells, purified, and transferred into ES cells or isolated oocytes. The transfected ES cells can then be injected into blastocysts to generate chimeras. The chimeras which transmit the mutation to their offspring are identified and selected. These animals can then be used as founder animals to obtain different animal lines, derived from breeding with chosen animals. Heterozygous animals can then be produced and further mated to generate a hybrid F1 cross. Further matings of the F1 heterozygotes produce the wild type, heterozygous and homozygous null mutants of 4E-BP1 (having both copies of the 4E-BP1 gene disrupted). The homozygous animals can then serve in a number of experiments. Non-limiting examples thereof include: the characterization of their phenotype, and a reconstitution of the 4E-BP1 activity by complementation by a non-endogenous copy of a wild type 4E-BP1 gene or mutant or variant 4E-BP1 gene. An animal (or cells derived therefrom) expressing a mutant form of 4E-BP1 gene (from human, for example) could be used to screen for compounds which modulate more specifically the mutant form of the 4E-BP1 gene.

The present invention therefore strongly indicates that 4E-BP1 is a regulator of fundamental cellular function in vivo. It is thus expected that this cellular function should occur across species. The presence of the 4E-BP1 gene and its conservation among species (human, mice, rats, fish and lower organisms such as Drosophila, support its essential role in physiology. Thus, the modulators of 4E-BP1-eIF-4E interaction identified by the methods and assays of the present invention should find a utility in the treatment of obesity and other metabolic diseases associated with lipid or glucose metabolism malfunction in humans and other animals.

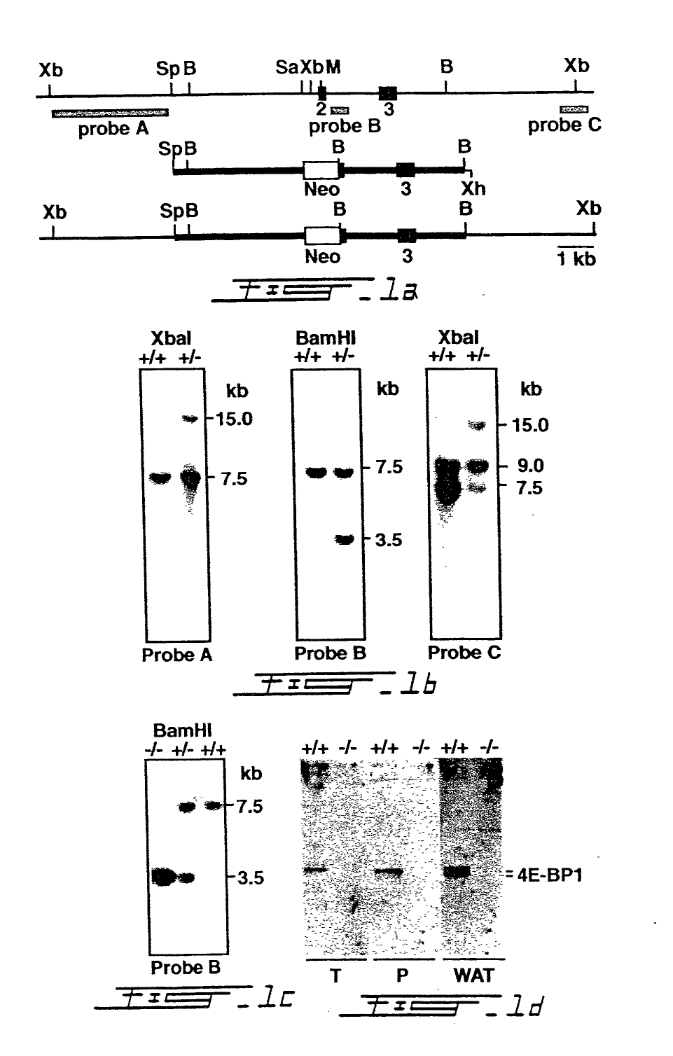

The Eif4ebp1 gene targeting vector was constructed to replace the splice acceptor site and the first 57 nucleotides of Eif4ebp1 exon 2 with the neomycin-resistance gene (FIG. 1a). Exon 2 encodes amino acids 47 to 108, which encompasses the binding domain for eIF4E. The disrupted portion of exon 2 encodes amino acids 47 to 66 of 4E-BP1. Following electroporation of linearized targeting vector DNA into the J1-129/SV embryonic stem (ES) cells, 800 G418 resistant colonies were analyzed by Southern blotting for homologous recombination. Two ES clones were found to contain a correct replacement (FIG. 1b). One of these clones, after injection into Balb/c blastocysts enabled germ line transmission. Heterozygous (Eif4ebp1^{+/-}) mice were then crossed to produce homozygous (Eif4ebp1^{-/-}) offspring, and the absence of 4E-BP1 expression in these animals was verified by Western blotting (FIG. 1c). Of note, no compensatory increase in 4E-BP2 or 4E-BP3 protein levels was observed (data not shown).

The number of Eif4ebp1^{-/-} offspring was consistent with the laws of Mendelian inheritance. Litters were of normal size, and the mice developed normally. After more than 2 years, no difference of lifespan was observed and the Eif4ebp1^{-/ -}mice show no evidence of illness or tumors according to a gross anatomical analysis. The mice have been followed until death. Blood glucose levels, however, were slightly lower in Eif4ebp1^{-/ -}mice (˜15%; Table 1). This hypoglycemia could not be explained by hyperinsulinemia, as plasma insulin levels were similar in wild-type and knockout mice (Table 1). Moreover, the amounts and plasma membrane translocation of the glucose transporters Glut-1 and Glut-4 were similar in wild-type and knockout mice (data not shown).

TABLE 1
Eif4ebp1 −/− mice have altered metabolic parameters

| | Wild-type | Knockout |
|---|---|---|
| Fed glycemia (mg/dl; n=5) | 105 ± 5 | 80 ± 6 * |
| Fasted glycemia (mg/dl; n=5) | 75 ± 4 | 63 ± 4 * |
| Insulin (ng/ml; n=5) | 1.7 ± 0.6 | 1.1 ± 0.5 |
| Leptin (ng/ml; n=8) | 8.7 ± 5.5 | 3.0 ± 1.8 * |
| Triglycerides (ng/ml; n=5) | 41.7 ± 2.0 | 65 ± 11 |

* P < 0.05 Data are presented as mean ± s.e.m. Statistical analysis was performed with a two-tailed, unpaired, Student's t test.

Routine histological examination of the major organs (e.g. liver and kidneys) revealed no abnormalities such as, for example, dysplastic tissue. However, a significant (P<0.05) decrease of approximately 10% in body weight was observed in homozygous males in comparison to their wild-type littermates (Table 2). The difference in weight was not due to hypophagia, as the food intake was the same for both wild-type and Eif4ebp1^{-/-} mice (data not shown). The decrease in body weight could be partially accounted for by a striking reduction of ˜60% in white adipose tissue (WAT) weight in Eif4ebp1^{-/-} males (Table 2). This size reduction was specific to adipose tissue, as heart (Table 2), and other tissues (data not shown) showed no significant weight difference between wild type and Eif4ebp1^{-/-} mice. Female Eif4ebp1^{-/-} mice exhibited a similar phenotype (Table 2). Consistent with the reduced adipose tissue mass, circulating leptin levels were decreased in Eif4ebp1^{-/-} mice (˜60%, Table 1). Triglycerides levels were also measured, but no statistically significant difference was observed between wild-type and knockout mice (Table 1). However it appears that there is a sexual dimorphism in this phenotype as the female Eif4ebp1^{-/-} mice do not show the same extent of decrease in their total body weight, even though their fat pads are also decreased in weight (˜50%, Table 1), as compared to their male counterparts.

TABLE 2
Eif4ebp1 knock-out mice have reduced adipose tissue mass
Weight (g)

| | Reproductive | Inguinal | Retroperitoneal | | |
|---|---|---|---|---|---|
| | Body | Fat-pad | fat-pad | fat-pad | Heart |
| Male | | | | | |
| Wild-type (n = 7) | 33.7 ± 1.1 | 0.75 ± 0.15 | 0.53 ± 0.06 | 0.33 ± 0.06 | 0.162 ± 0.008 |
| Knock-out (n = 6) | 30.6 ± 0.5 | 0.26 ± 0.05 | 0.22 ± 0.06 | 0.12 ± 0.04 | 0.166 ± 0.004 |
| % of wild type | 91% | 35% | 42% | 36% | 102% |
| | P < 0.05 | P < 0.05 | P < 0.01 | P < 0.05 | P = 0.70 |
| male | | | | | |
| Wild-type (n = 8) | 30.0 ± 0.5 | N.D. | 0.53 ± 0.03 | 0.40 ± 0.06 | N.D. |
| Knock-out (n = 8) | 27.0 ± 0.3 | N.D. | 0.28 ± 0.02 | 0.21 ± 0.04 | N.D. |
| % of wild type | 90% | | 53% | 53% | |
| | P = 0.08 | | P < 0.01 | P < 0.05 | |

Data are presented as means ± s.e.m. The mice studied here were 10-12 months old. Statistical analysis was performed with a two-tailed, unpaired, Student's t test.

In view of the reduced adipose tissue in Eif4ebp1^{-/-} mice, it was of interest to determine whether Eif4ebp1 disruption might affect glucose homeostasis and metabolic rate. To investigate this possibility, an insulin tolerance test was performed. Briefly, the blood glucose concentration of five fed male mice was determined (16:30). Five male mice were fasted for 6 h (3:00-9:00 AM), and insulin (Eli-Lilly) was injected (0.4 U/kg) intraperitoneally. Blood was collected serially from retro-orbital sinus or tail vein under anesthesia and blood glucose levels were measured; the insulin tolerance test was performed twice and the mean and standard deviation from the mean calculated.

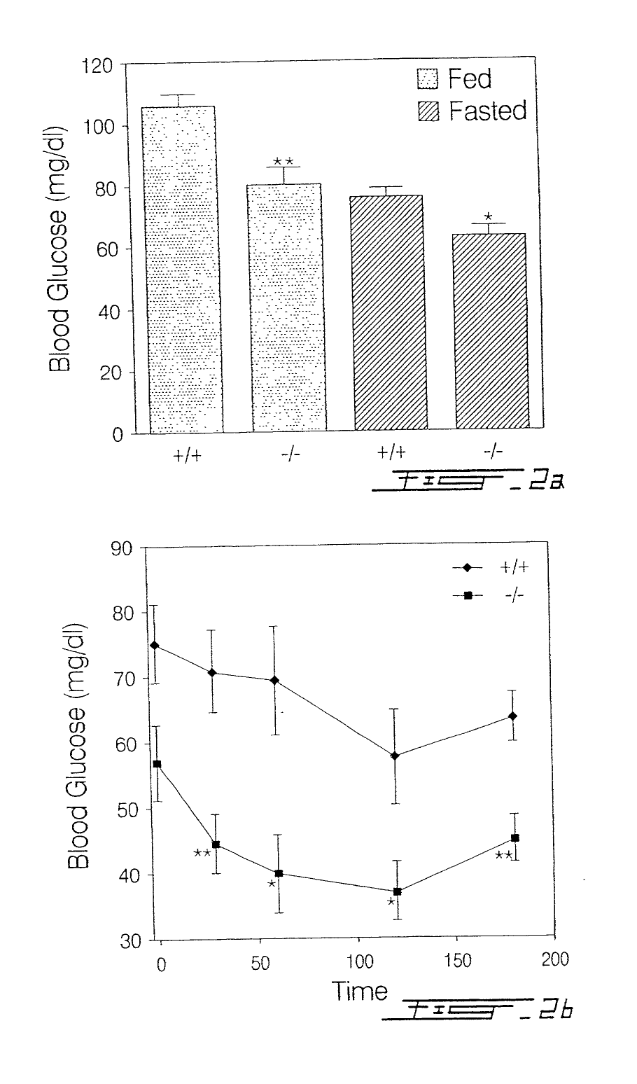

In the fed state (FIG. 2a), the basal level of glucose after fasting was lower by ˜20% in Eif4ebp1^{-/-} mice (FIG. 2b, t=0) as compared to their wild-type littermates. This ratio was maintained following insulin treatment and during recovery (FIG. 2b). Thus, the 4E-BP1^{-/-} mice are not diabetic. This result indicates that the regulation of glucose uptake and metabolism in response to insulin is not altered in Eif4ebp1^{-/-} mice, but rather that some constitutive change in glucose homeostasis has occurred in the Eif4ebp1^{-/-} mice. A glucose tolerance test was also performed, but no significant difference was observed between wild-type and knockout animals (data not shown).

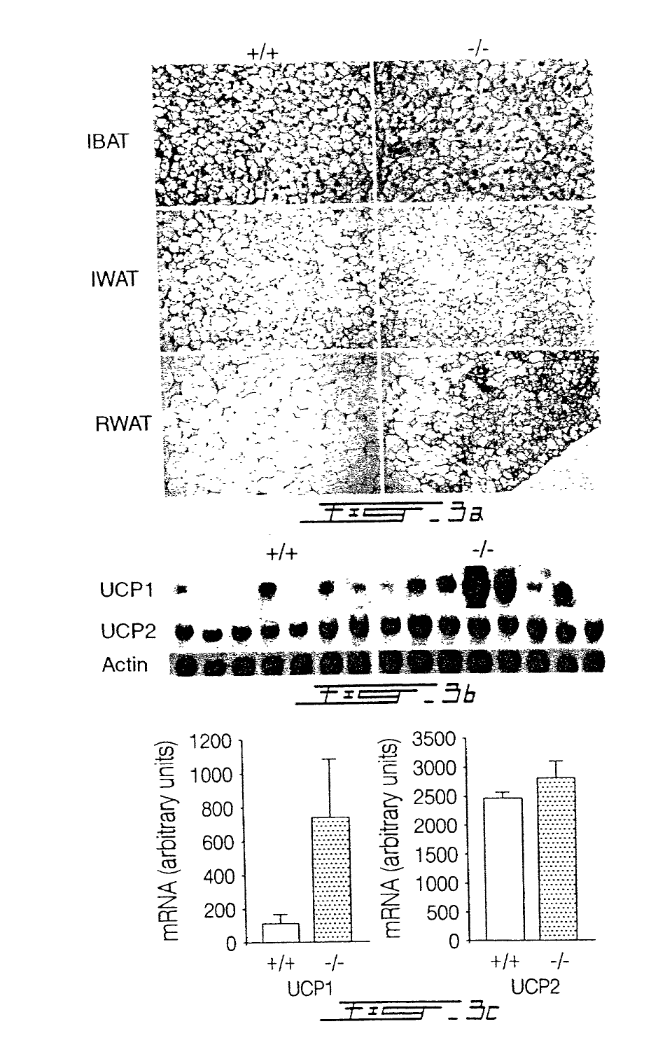

To further characterize the adipose tissue phenotype, histological sections of white adipose tissue (WAT) and interscapular brown adipose tissue (IBAT) were examined. The inguinal and retroperitoneal WAT (IWAT and RWAT) of Eif4ebp1^{-/-} mice displayed a striking increase in the number of multilocular adipocytes, which are characteristic of brown adipose tissue (FIG. 3a). Furthermore, Eif4ebp1^{-/-} IBAT displays smaller lipid droplets (FIG. 3a). These histological observations could be explained if energy expenditure was increased in the knockout mice. Consequently, the resting metabolic rate (RMR) of Eif4ebp1^{-/-} mice was examined and a significant increase in the males (˜15%; Table 3) was observed. The difference between the RMR of Eif4ebp1^{-/-} and wild type female mice was not statistically significant (Table 3).

At the molecular level, a major difference between WAT and BAT is the expression of an uncoupling protein (UCP1), which uncouples oxidative phosphorylation in the BAT inner mitochondrial membrane (Boss et al., 1998). UCP1 is responsible for the increased thermogenesis associated with brown adipocytes (Lowell et al., 1993 and Enerbäck et al., 1997) and its overexpression prevents genetic obesity in mice (Kopecky et al., 1995). The expression levels of UCP1 and UCP2 mRNAs in IWAT from wild-type and Eif4ebp1^{-/-} mice were thus examined (FIG. 3b). Consistent with the increased number of multilocular adipocytes, the mRNA expression of UCP1, but not UCP2, is increased ˜6 fold in IWAT (FIG. 3c). Thus, the histological, physiological and molecular features of brown adipocytes are all clearly apparent in the white adipose tissue of Eif4ebp1 knockout mice.

TABLE 3
Analysis of Eif4ebp1 knock-out mice metabolic rate

| | Metabolic rate |
|---|---|
| | (ml O₂/kg/hr) |
| Male | |
| Wild type (n=9) | 2998 ± 186 |
| Knock-out (n=8) | 3469 ± 185* |
| Female | |
| Wild type (n=7) | 3182 ± 152 |
| Knock-out (n=8) | 3307 ± 136 |

* P < 0.05 Results Data are presented as means ± s.e.m. Statistical analysis was performed with a two-tailed, unpaired, Student's t test.

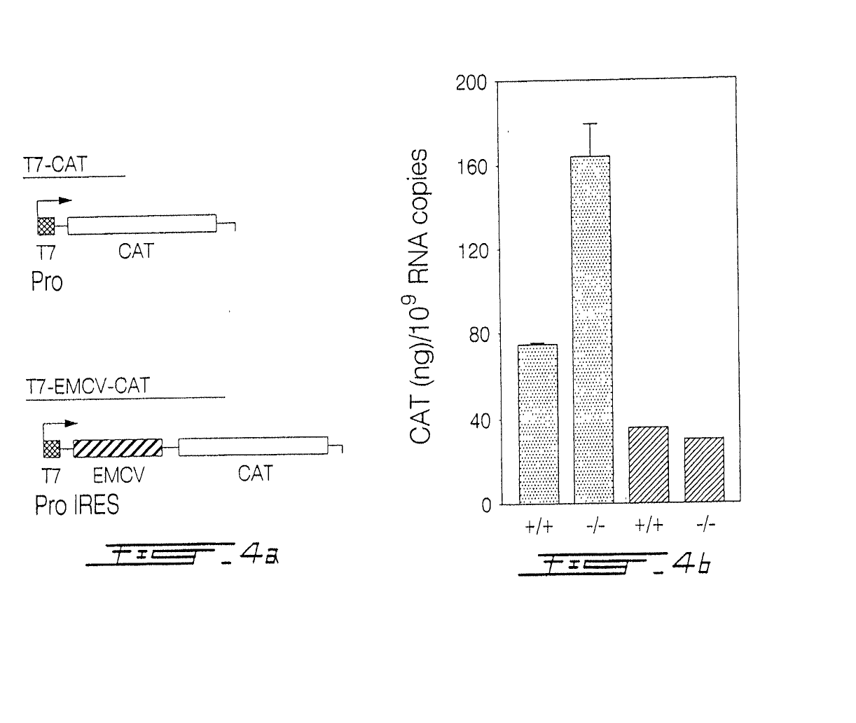

Thus, the data herein presented reveal an unanticipated role for 4E-BP1 in the regulation of fat metabolism. The mechanism explaining this regulation is not immediately clear, however. The assessment as to whether the unanticipated role of 4E-BP1 in the regulation of metabolism is through a modulation of protein synthesis was examined in primary embryo fibroblasts (MEFs) derived from wild type and Eif4ebp1^{-/-} mice. General translation rates were examined by metabolic labelling with ³H[leucine], but no significant change was observed between wild-type and Eif4ebp1^{-/-} MEFs (data not shown). Because 4E-BP1 inhibits cap-dependent, but not cap-independent translation (Pause et al., 1994, supra), the effect of Eif4ebp1 disruption on both translation modes was examined. Thus, the expression of chloramphenicol acetyltransferase (CAT) from a construct in which translation is cap-dependent (T7-CAT) was compared to that in which translation is directed by an Internal Ribosome Entry Site (IRES) from which translation is cap-independent (T7-EMCV-CAT) (FIG. 4a). Given that elimination of 4E-BP1 should result in increased availability of eIF4E for eIF4F formation, a preferential enhancement of cap-dependent translation is expected in 4E-BP1^{-/-} MEFs relative to its translation in wild-type MEFs. Consistent with this prediction, the quantity of CAT protein production per RNA molecule was 117% higher in Eif4ebp1^{-/-} MEFs than in wild-type MEFs (FIG. 4b). In contrast, CAT expression from the IRES-dependent EMCV-CAT vector was decreased only slightly (15%; FIG. 4b). To ensure that the differences observed in this experiment reflect changes in translation rather than differences in RNA levels, an RNA quantitation using a real time detection PCR method was performed (data not shown), thus yielding a quantity of CAT protein per 10⁹ RNA copies (FIG. 4b). Taken together, FIGS. 4a and 4b show that the elimination of 4E-BP1 resulted in enhancement of cap-dependent translation initiation.

Since elimination of 4E-BP1 causes an increase in the amount of eIF4E that is available for incorporation into eIF4F, and that changes in translation caused by eIF4E overexpression are associated with changes in cell growth, the growth properties of wild-type and Eif4ebp1^{-/-} primary mouse embryonic fibroblasts (MEFs) were examined. As shown in FIG. 4c, Eif4ebp1^{-/-} MEFs exhibited faster growth rates (10-20%) than wild-type MEFs (FIG. 4). This was also evident when cells were kept for longer periods of time in culture (data not shown).

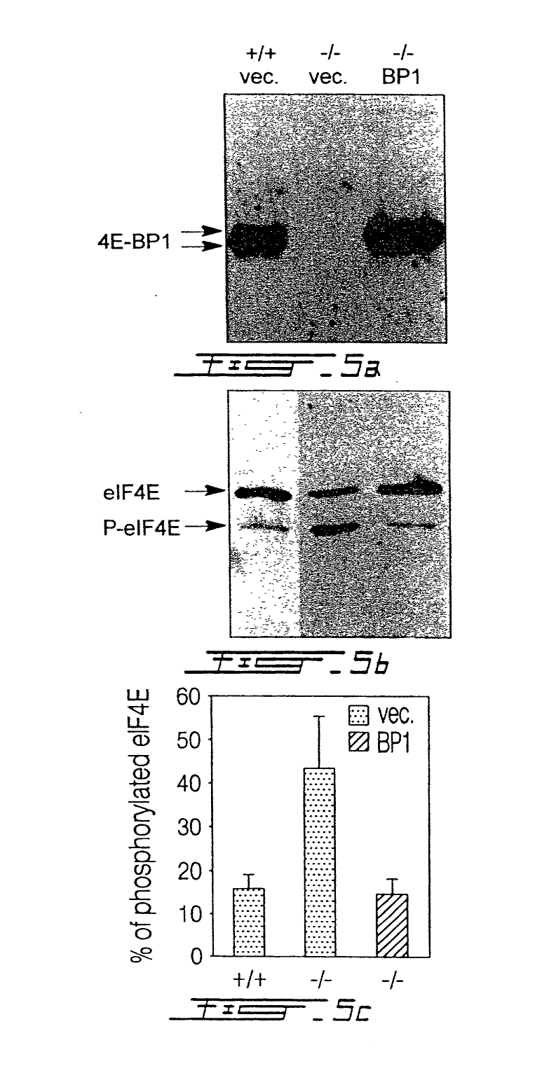

Numerous studies have reported a positive correlation between cell growth and translation rates and eIF4E phosphorylation state. Morevover, the in vitro phosphorylation of eIF4E by two kinases (Mnkl and PKC) has been shown to be inhibited by its binding to 4E-BP1. Eif4ebp1^{-/-} MEFs were used to study the status of eIF4E phosphorylation, which is known to correlate with translation rates and cell growth status (Gingras et al., 1999). eIF4E is phosphorylated by the docking of Mnk1, a serine/threonine kinase, on eIF4G is prevented by the binding of 4E-BP1 to eIF4E (Pyronnet et al., 1999). The deletion of 4E-BP1 in the mouse is thus expected to lead to an increase in eIF4E phosphorylation. The effects of 4E-BP1 deletion on eIF4E phosphorylation were analyzed by isoelectric focusing (FIG. 5b). Indeed, eIF4E phosphorylation in MEFs was increased from 16% in wild-type MEFs to 44% in Eif4ebp1^{-/-} cells (FIG. 5c). To confirm that the increase in eIF4E phosphorylation in Eif4ebp1^{-/-} cells was caused by the absence of 4E-BP1, Eif4ebp1^{-/-} MEFs were transfected with a 4E-BP1 expression vector (a knock-in approach) (FIG. 5a). Consistent with the direct role of 4E-BP1 in affecting the phosphorylation status of eIF-4E, the expression of 4E-BP1 in the Eif4ebp1^{-/-} cells led to a decrease (˜66%) in eIF4E phosphorylation (FIGS. 5b and 5c). Thus, these data provide the evidence that 4E-BP1 can also regulate eIF4E phosphorylation.

Taken together, the data herein presented indicate that 4E-BP1 is a novel mediator of energy homeostasis in mammals. In addition, they identify translation control and more particularly cap-dependent translation as a key process in energy homeostasis in animals. While not being limited to a particular theory, the most likely underlying mechanism to explain these results is the up-regulation of eIF4E activity, which would then specifically affect the translation of mRNAs involved in brown adipocytes activation and function. One such candidate mRNA is the uncoupling protein-1 (UCP1), a specific marker of brown adipocytes. However, the increase in UCP1 mRNA expression cannot be directly linked to the function of eIF4E in translation initiation. Instead, eIF4E might stimulate the translation of an mRNA encoding a factor which is involved in brown adipocyte differentiation, mitochondrial biogenesis, or in the up-regulation UCP1 expression. The molecular determinants regulating brown fat cells differentiation are still poorly characterized. One possible candidate is the peroxisome proliferator-activated receptor y (PPARy), which can specifically transactivate the UCP1 promoter in brown adipocytes (Wu et al., 1999a). However, PPARy is also expressed in white fat cells and thus cannot explain the specificity of UCP1 induction. Another candidate is the PPARY coactivator 1 (PGC1), a coactivator of nuclear receptors involved in adaptative thermogenesis and mitochondrial biogenesis (Wu et al., 1999b). PGC1 activates UCP1 expression when ectopically expressed in 3T3-F442A preadipocytes, and UCP2 expression when expressed in C2C12 myotubes. It would thus be interesting to investigate whether the expression of PGC1 mRNA is under translational control. Finally, it is also possible that eIF4E affects the expression of a protein involved in the signaling pathway activating the UCP1 promoter.

An Eif4ebp1 knockout mouse has been reported previously, but the features of reduced fat tissue and increase multilocular adipocytes in WAT described in the present study were not reported or suggested therein, although a reduction in the body weight of male mice was noted (Blackshear et al., 1997). A plausible explanation for this discrepancy might be the different mouse strains used to backcross the F0 mice. While Balb/c mice were used herein for crossing to the 129 strain, Blackshear et al. used C57BL6/J. There are numerous reports showing strain-dependent phenotypic changes in mice, especially when assessing metabolic disorders (see, for example, Ewart-Toland et al., 1999; Surwit et al., 1995, Coleman et al., 1973; and Hummel et al., 1972). Moreover, the emergence of brown adipocytes in white fat has been shown to be under a complex genetic control (Guerra et al. 1998), which might explain the fluctuations in UCP1 expression levels (FIG. 3b). Consequently, the Eif4ebp1^{-/-} mutation is being transferred to the Balb/c strain originally used to backcross the 129 chimera. In an inbred background, the Eif4ebp1^{-/-} phenotype should be more readily amenable to metabolic studies. In such a genetic background based on Balb/c (e.g. an inbred background) variability would be minimized. The implication of cap-dependent translation is glucose metabolism.

A large number of studies on cells in culture have provided evidence that eIF4E plays an important role in the control of cell growth. The conclusions from the earlier studies are supported by the present results, which provide evidence that translation initiation in animals plays an important role in cell growth and body metabolism.

The finding that 4E-BP1 is implicated in the control of fat tissue growth, metabolism, and glucose homeostasis is of pharmacological value, as specific modulation of the 4E-BP1-eIF-4E interaction, as well as the modulation of the formation of the eIF-4F preinitiation complex and of the level of eIF-4E complex to eIF-4G1, could be used to modulate fat and glucose metabolism, for example. This possibility is particularly intriguing in light of the fact that 4E-BP1 elimination is not deleterious to mice health.

Furthermore, the present invention, having identified translation initiation through eIF4E and its association with eIF-4G as a biochemical pathway involved in metabolism in vivo, provides numerous assays and methods to screen and identify metabolism modulators and especially fat and glucose metabolism modulators.

Two functional 4E-BP1 homologs, 4E-BP2 and 4E-BP3, exist in mammals (Pause et al., 1994; and Poulin et al., 1998). Although no functional differences have been reported among them, their tissue distribution differs (Poulin et al., 1998; and Tsukiyama-Kohara et al., 1996). For example, 4E-BP1 is more abundant in WAT as compared to the other homologs (not shown and Hu et al., 1994; and Lin et al., 1996). It is conceivable that in Eif4ebp1^{-/-} mice, the presence of 4E-BP2 and 4E-BP3 may attenuate a phenotype that would have been observed by the loss of all three proteins. Thus, a double, and perhaps a triple knockout, might exhibit a more severe phenotype in WAT reduction, and might also show additional phenotypic changes not observed in Eif4ebp1^{-/-}. The actual phenotype of 4E-BP2, 4E-BP3 single knockout animals or of double or triple knockouts awaits formal testing.

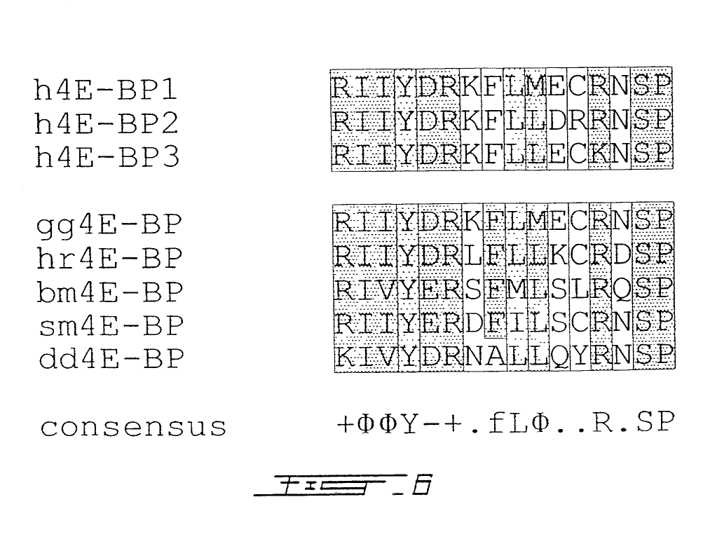

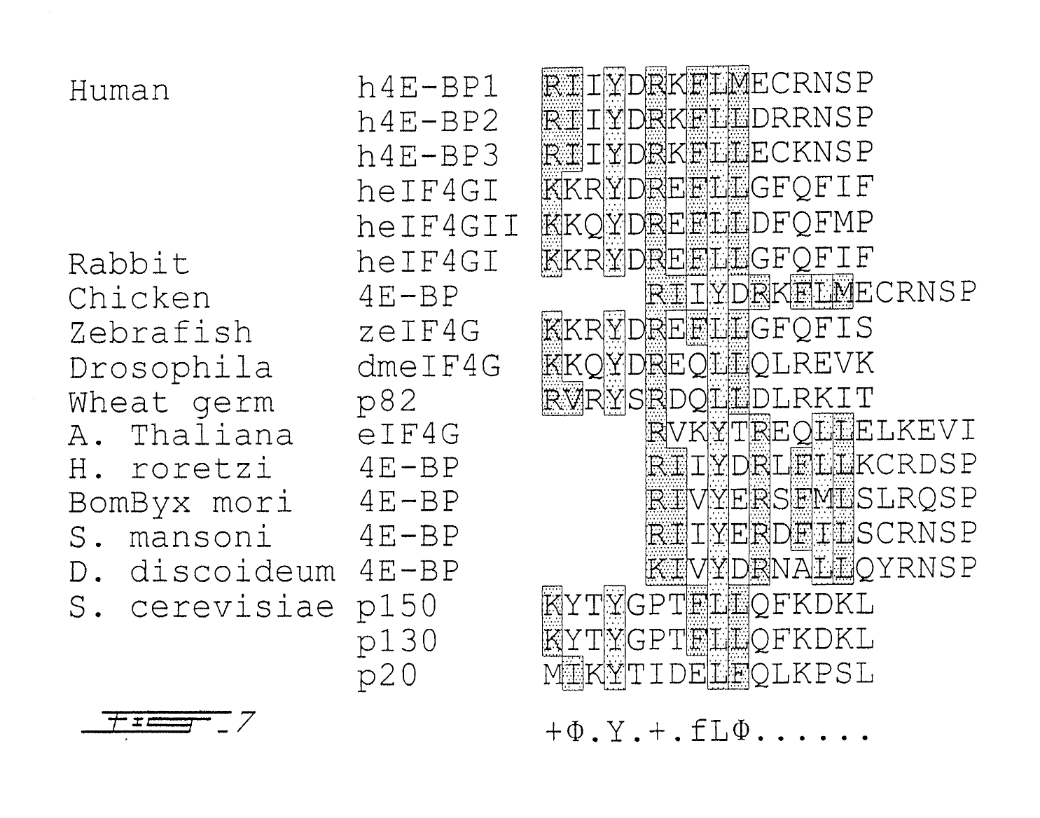

As seen in FIGS. 6 and 7, the eIF4E binding sites (or eIF4E interaction domains) of numerous protein from evolutionarily distant organisms show a significant homology/identity. In addition, the sequences of rat and mouse 4E-BP1, 4E-BP2 and 4E-BP3 are 100% identical to those of the human in the region presented here. Indeed, consensus sequences which retain their eIF4E binding activity are provided. For example, a consensus 4E-binding sites of 4E-BPs is +φφY−+xF/AφφxxRxSP wherein + and − refer to a charged amino acid; φ is a hydrophobic amino acid; x is any amino acid; and the capital letters refer to the known one letter code for amino acids. Preferably, the consensus sequence has the sequence +φφY−+xfLφxxRxSP, wherein f refers to a preferred but apparently the non-essential amino acid Phe (the rest is as for the previous consensus sequence). In yet another embodiment, the 4E-binding consensus sequence has the sequence +φxxYx+Xfφφ or YxxxxLφ. Conversely, they could be used to design eIF-4E or negative regulators thereof, which no longer interact or show lower affinities. These consensus sequences could be used as eIF4E sequestering agents or as starting points to design other eIF4E sequestering agents.

Of note, recombinant peptides derived from 4E-BP1 and eIF-4GII have been shown to inhibit translation of mRNAs (Marcotrigiano et al., 1999, Molecul. Cell 3:707-716).

The present invention is illustrated in further detail by the following non-limiting examples.

### EXAMPLE 1

### Generation of Eif4ebp1 Deficient Mice

Fragments (4 kb; Spe I-Sal I) and (3.5 kb; MscI-BamHI) of the murine Eif4ebp1 gene were ligated to the 5′ and 3′ ends of the pGK-Neo vector (polyA-) to construct the targeti/ng vector for gene disruption. The DNA was digested with Spe I and Xhol, purified with LGT agarose (FMC) and electroporated into ES cells (129/sv) with a Gene Pulser (Bio-Rad). Cells were selected with G418, as described previously. G418 resistant clones were screened for correct targeting by Southern blotting using probe a (FIG. 1a, b), and positives clones were confirmed with probe c (FIG. 1a, b). Properly targeted hemizygous ES cells were injected into Balb/c blastocysts and chimera mice were backcrossed to Balb/c mice to generate Eif4ebp1^{+/-} mice. Following mating of the heterozygous mice, genomic Southern blotting using probe a or b was performed to genotype the progeny mice (FIG. 1c). The absence of 4E-BP1 in mouse tissue was confirmed by Northern blot analysis using a portion of exon 2 (SmaI-Mscl, nt 142-203 of the coding region) as a probe (data not shown), and Western blotting (FIG. 1c), using anti-4E-BP1 antibody. MEF cells were prepared from 14 day old embryos, as described previously.

### EXAMPLE 2

### Plasmids and Transfections

4E-BP1 expression vector was constructed as follows: the mouse 4E-BP1 cDNA was cloned by RT-PCR using sense primer 5′- TGCAGGAGACATGTCG-3′ and anti-sense primer 5′-ACAGTTTGAGATGGAC-3′, with SUPERSCRIPTII (GIBCO-BRL) and Pfu polymerase (TOYOBO). It was sequenced and subcloned under the control of a CAG promoter (AG promoter with CMV-IE enhancer). A puromycin resistant cassette was derived from the pBabe-PURO vector and introduced into the 4E-BP1 expression vector, which was transfected (4 mg) into MEF cells (6 cm dishes) with Lipofectin (Gibco-BRL). T7-CAT was described previously and EMCV CAT was kindly provided by Dr. Sung-Key Jang (POHANG Institute of Science and Technology, Korea).

### EXAMPLE 3

### Isoelectric Focusing of eIF4E

One-dimensional vertical slab isoelectric focusing was carried out as described previously using a protean II minigel apparatus (Bio-Rad). Proteins were focused on a 5% acrylamide gel containing 9.5M urea, 3% ampholine pH4.5-5.5, 1% ampholine pH3.5-10, 2% CHAPS). Histidine (10 mM) was used as the cathode buffer and glutamic acid (50 mM) was used as the anode buffer. Focusing was performed for 3 h at 500-750 V, followed by transfer of proteins to a polyvinylidene fluoride membrane (Immobilon P, Millipore). Filters were probed with anti-eIF-4E rabbit polyclonal antibody as described previously (Frederickson et al., 1991).

### EXAMPLE 4

### Expression of Exogenous RNA and Their Quantitation

MEF cells were transfected with T7-CAT and T7-EMCV-CAT plasmids by Lipofectin, followed by infection with a recombinant vaccinia virus expressing the T7 RNA polymerase gene (LOT7-1 RVV) as described previously (Takeuchi et al., 1999). RNA quantitation was performed by a real time detection PCR method using a sense primer (5′-GGGTGAGTTTCACCAGTTTTGA-3′), an anti-sense primer (5′-CCACTCATCGCAGTACTGTTGT-3′), and a probe (5′(FAM)-CAATATGGACAACTTCTTCGCCCC-(TAMRA)3′), as described previously (Yasui et al., 1998). Expressed CAT protein was measured by CAT-ELISA (Roche).

### EXAMPLE 5

### Oxygen Consumption

Oxygen consumption (VO₂) was simultaneously determined for 4 mice per experiment in an Oxymax metabolic chamber (Columbus Instruments). Individual mice (18 to 24 weeks old) were placed in a chamber with an airflow of 0.5 L/min. Ambient temperature was maintained between 24.5 and 25.5° C. Experiments for male mice were performed between 12:00 pm and 3:00 pm, and for females mice between 3:00 pm and 6:00 pm. Mice were placed into the chambers one hour before beginning the experiment to reduce anxiety. Five reading were taken at ten minutes intervals over the next 50 minutes and averaged.

### EXAMPLE 6

### Metabolic Parameters

Male animals were either fed ad libitum (Fed) or fasted for 6 h (Fasted). Serum glucose levels were measured using a One Touch Basic glucometer (Lifescan Canada Ltd.). Fed insulin levels in serum were measured using a radioimmunoassay (Linco). Fed serum leptin levels were measured by ELISA (R&D Systems). Fed serum triglycerides levels were measured using a triglycerides detection kit (WAKO).

### CONCLUSION

These findings show that cap-dependent translation significantly regulates energy homeostasis, and glucose and fat metabolism in animals. More particularly, it identifies the sequestration of eIF-4E as a key determinant in these critical pathways. Furthermore, 4E-BP1 is shown to be an important regulator of body metabolism as a consequence of its function as a repressor of translation.

Although the present invention has been described hereinabove by way of preferred embodiments thereof, it can be modified, without departing from the spirit and nature of the subject invention as defined in the appended claims.

### REFERENCES

1. Gingras, A. -C., Raught, B. & Sonenberg, N. eIF-4 Initiation Factors: Effectors of mRNA Recruitment to Ribosomes and Regulators of Translation. Annu. Rev. Biochem. 68(1999).

2. Pause, A. et aL. Insulin-dependent stimulation of protein synthesis by phosphorylation of a regulator of 5′-cap function. Nature 371, 762-767 (1994).

3. Poulin, F., Gingras, A. C., Olsen, H., Chevalier, S. & Sonenberg, N. 4E-BP3, a new member of the eukaryotic initiation factor 4E-binding protein family. J. Biol. Chem. 273, 14002-14007 (1998).

4. Tsukiyama-Kohara, K. et al. Tissue distribution, genomic structure, and chromosome mapping of mouse and human eukaryotic initiation factor 4E-binding proteins 1 and 2. Genomics 38, 353-363 (1996).

5. Takeuchi, T. et al. Real-time detection system for quantitation of Hepatatis C virus genome. Gastroenterology 116, 636-642 (1999).

6. Lazaris-Karatzas, A. et al. Ras mediates translation initiation factor 4E-induced malignant transformation. Genes Dev 6, 1631-1642 (1992).

7. Minich, W. B., Balasta, M. L., Goss, D. J. & Rhoads, R. E. Chromatographic resolution of in vivo phosphorylated and nonphosphorylated eukaryotic translation initiation factor eIF-4E: increased cap affinity of the phosphorylated form. Proc. Natl. Acad. Sci. USA 91, 7668-7672 (1994).

8. Wang, X. et al. The phosphorylation of eukaryotic initiation factor eIF4E in response to phorbol esters, cell stresses, and cytokines is mediated by distinct MAP kinase pathways. J. Biol. Chem. 273, 9373-9377 (1998).

9. Whalen, S. G. et a/. Phosphorylation of eIF-4E on serine 209 by protein kinase C is inhibited by the translational repressors, 4E-binding proteins. J Biol Chem 271, 11831-11837 (1996).

10. Sonenberg, N. mRNA 5° Cap-binding Protein eIF4E and Control of Cell Growth. in Translational Control (eds. Hershey, J. W .B., Mathews, M. B. & Sonenberg, N.) 245- 270 (Cold Spring Harbor Laboratory Press, Cold Spring Harbor, N.Y., 1996).

11. Jain, R. G., Andrews, L. G., McGowan, K. M., Pekala, P. H. & Keene, J. D. Ectopic expression of Hel-N1, an RNA-binding protein, increases glucose transporter (GLUT1) expression in 3T3-L1 adipocytes. Mol. Cell. Biol. 17, 954-962 (1997).

12. Lin, T.A. et al. PHAS-I as a link between mitogen-activated protein kinase and translation initiation. Science 266, 653-656 (1994).

13. Blackshear, P. J., Stumpo, D. J., Carballo, E. & Lawrence, J. C., Jr. Disruption of the Gene Encoding the Mitogen-regulated Translational Modulator PHAS-I in Mice. J. Biol. Chem. 272, 31510-31514 (1997).

14. Sibilia, M. & Wagner, E. F. Strain-dependent epithelial defects in mice lacking the EGF receptor. Science 269, 234-238 (1995).

15. Lazaris-Karatzas, A., Montine, K. S. & Sonenberg, N. Malignant transformation by a eukaryotic initiation factor subunit that binds to mRNA 5′ cap. Nature 345, 544-547 (1990).

16. Rousseau, D., Gingras, A. -C., Pause, A. & Sonenberg, N. The eIF4E-binding proteins 1 and 2 are negative regulators of cell growth. Oncogene 13, 2415-2420 (1996).

17. Ramírez-Solis, R., Davis, A. C. & Bradley, A. Gene targeting in embryonic stem cells. Methods Enzymol. 225, 855-878 (1993).

18. Gingras, A. -C., Svitkin, Y., Belsham, G. J., Pause, A. & Sonenberg, N. Activation of the translational suppressor 4E-BP1 following infection with encephalomyocarditis virus and poliovirus. Proc. Natl. Acad. Sci. USA 93, 5578-5583 (1996).

19. Robertson, E. J. Embryo-derived stem cell lines. in Tetracarcinomas and embryonic stem cells: A practical approach (ed. Robertson, E.J.) 71-117 (IRL Press, Oxford, 1987).

20. Niwa, H., Yamamura, K. & Miyazaki, J. Efficient selection for high-expression transfectants with a novel eukaryotic vector. Gene 108, 193-199 (1991).

21. Morgenstern, J. P. & Land, H. Advanced mammalian gene transfer: high titre retroviral vectors with multiple drug selection markers and a complementary helper-free packaging cell line. Nucleic Acids Res. 18, 3587-3596 (1990).

22. Tsukiyama-Kohara, K., lizuka, N., Kohara, M. & Nomoto, A. Internal ribosome entry site within hepatitis C virus RNA. J. Virol. 66, 1476-1483 (1992).

23. Frederickson, R. M., Montine, K. S. & Sonenberg, N. Phosphorylation of eukaryotic translation initiation factor 4E is increased in Src-transformed cell lines. Mol. Cell. Biol. 11, 2896-2900 (1991).

24. Yasui, K. et al. The native form and maturation process of hepatitis C virus core protein. J. Virol. 72, 6048-6055 (1998).

24. Jacobson, A. “Poly(A) metabolism and translation: The closed loop model.” In Translational Control, eds. J. W. B. Hershey, M. B. Mathews, and N. Sonenberg. 451-480. Cold Spring Harbor, N.Y.: Cold Spring Harbor Laboratory Press, 1996.

25. Altmann, M., Schmitz, N., Berset, C., and Trachsel, H. (1997). A novel inhibitor of cap-dependent translation initiation in yeast—p20 competes with eIF4G for binding to eIF-4E. EMBO J. 16, 1114-1121.

26. Haghighat, A., Mader, S., Pause, A., and Sonenberg, N. (1995). Repression of cap-dependent translation by 4E-binding protein 1: competition with p220 for binding to eukaryotic initiation factor—4E. EMBO J. 14, 5701-5709.

27. Mader, S., Lee, H., Pause, A., and Sonenberg, N. (1995). The translation initiation factor eIF-4E binds to a common motif shared by the translation factor eIF-4G and the translational repressors 4E-binding proteins. Molec. Cell. Biol. 15, 4990-4997.

28. Sonenberg, N. and Gingras, A. -C. (1998). The mRNA 5′ cap-binding protein eIF4E and control of cell growth. Curr. Opin. Cell Biol. 10, 268-275.

29. Marcotrigiano, J., Gingras, A. -C., Sonenberg, N., and Burley, S. K. (1997). Cocrystal structure of the messenger RNA 5′ cap-binding protein (eIF4E) bound to 7-methyl-GDP. Cell 89, 951-961.

30. Matsuo, H., Li, H., McGuire, A. M., Fletcher, C. M., Gingras, A. -C., Sonenberg, N., and Wagner, G. (1997). Structure of translation factor eIF-4E bound to m7GDP and interaction with 4E-binding protein. Nature Struct. Biol. 4, 717-724.

31. Fletcher, C. M., McGuire, A. M., Gingras, A. -C., Li, H., Matsuo, H., Sonenberg, N., and Wagner, G. (1998). 4E binding proteins inhibit the translation factor eIF4E without folded structure. Biochemistry 37, 9-15.

32. Boss, O., Muzzin, P. & Giacobino, J. P. The uncoupling proteins, a review. Eur J Endocrinol 139, 1-9 (1998).

33. Lowell, B. B. et al. Development of obesity in transgenic mice after genetic ablation of brown adipose tissue. Nature 366, 740-742 (1993).

34. Enerbäck, S. et al. Mice lacking mitochondrial uncoupling protein are cold-sensitive but not obese. Nature 387, 90-94 (1997).

35. Kopecky, J., Clarke, G., Enerbäck, S., Spiegelman, B. & Kozak, L. P. Expression of the mitochondrial uncoupling protein gene from the aP2 gene promoter prevents genetic obesity. J. Clin. Invest. 96, 2914-2923 (1995).

36. Pyronnet, S. et al. Human eukaryotic translation initiation factor 4G (eIF4G) recruits Mnk1 to phosphorylate eIF4E. EMBO J. 18, 270-279 (1999).

37. Wu, Z., Puigserver, P. & Spiegelman, B. M. Transcriptional activation of adipogenesis. Curr. Opin. Cell Biol. 11, 689-694 (1999).

38. Wu, Z. et al. Mechanisms controlling mitochondrial biogenesis and respiration through the thermogenic coactivator PGC-1. Cell 98, 115-124 (1999).

39. Ewart-Toland, A., Mounzih, K., Qiu, J. & Chehab, F. F. Effect of the genetic background on the reproduction of leptin-deficient obese mice. Endocrinology 140, 732-738 (1999).

40. Surwit, R. S. et aL Differential effects of fat and sucrose on the development of obesity and diabetes in C57BL/6J and A/J mice. Metabolism 44, 645-651 (1995).

41. Coleman, D. L. & Hummel, K. P. The influence of genetic background on the expression of the obese (Ob) gene in the mouse. 6i Diabetologia 9, 287-293 (1973).

42. Hummel, K. P., Coleman, D. L. & Lane, P. W. The influence of genetic background on expression of mutations at the diabetes locus in the mouse. l. C57BL-KsJ and C57BL-6J strains. Biochem. Genet. 7,1-13 (1972).

43. Guerra, C., Koza, R. A., Yamashita, H., Walsh, K. & Kozak, L. P. Emergence of brown adipocytes in white fat in mice is under genetic control. Effects on body weight and adiposity. J. Clin. Invest 102, 412-420 (1998).

44. Hu, C., Pang, S., Kong, X., Velleca, M. & Lawrence, J. C., Jr. Molecular cloning and tissue distribution of PHAS-I, an intracellular target for insulin and growth factors. Proc. Natl Acad. Sci. USA 91, 3730-3734 (1994).

45. Lin, T. A. & Lawrence, J. C., Jr. Control of the translational regulators PHAS-I and PHAS-II by insulin and cAMP in 3T3-L1 adipocytes. J. Biol. Chem. 271, 30199 30204 (1996).

What is claimed is:

1. A non-human transgenic animal whose germ cells and somatic cells contain a knockout mutation in DNA encoding 4E-BP1, and wherein said transgenic animal shows a phenotype of an altered glucose and/or fat metabolism as compared to a control animal.

2. The transgenic animal of claim 1, wherein said animal is a mammal.

3. The mammal of claim 2, wherein said mammal is a mouse.

4. A cell line derived from the non-human transgenic animal of claim 1.

5. A cell line derived from the mouse of claim 3.

6. A method of producing a non-human transgenic animal, in which at least some cells thereof contain an altered gene encoding an altered 4E-BP1, said altered gene having been targeted to disrupt the wild type 4E-BP1 gene in said transgenic animal, said method comprising:
a) providing an altered gene encoding the altered form of 4E-BP1 and designed to target and disrupt said wild type 4E-BP1 gene of an embryonic stem cells (ES) of said animal;
b) introducing said altered gene in said ES cells;
c) selecting ES cells in which said altered 4E-BP1 gene has disrupted said wild type 4E-BP1 gene;
d) injecting said selected ES cells of c) into blastocysts;
e) implanting said blastocysts of d) in a pseudopregnant animal; and
f) producing a transgenic animal having at least some cells having said altered 4E-BP1 gene encoding said altered 4E-P1.

7. A method of identifying an agent which modulates glucose or fat metabolism in vivo comprising:
a) administering an agent suspected of being a modulator of cap-dependent translation in an animal;
b) measuring glucose and/or lipid levels in the animal of step a) and comparing same with that of a control animal, not having been administered said agent, wherein a difference in glucose and/or lipid levels of the animal of step a) as compared to that of the control animal identifies said agent as a modulator of glucose or fat metabolism in vivo.

8. The method of claim 7, wherein said positive modulator of cap-dependent translation is a. modulator of the level or of the activity of 4E-BP1.

9. The method of claim 8, wherein said agent increases and/or strengthens the interaction of 4E-BP1 with eIF-4E.

10. The method of claim 7, wherein said agent decreases and/or weakens the interaction of 4E-BP1 with eIF-4E.

11. The method of claim 7, wherein said agent decreases the amount of eIF-4F pre-initiation complex, thereby decreasing the translation of mRNAs implicated in glucose or lipid metabolism.

12. The method of claim 11, wherein said decrease in the amount of eIF-4F pre-initiation complex involves a sequestration of eIF-4E in a complex with an eIF-4E sequestering agent.

13. The method of claim 12, wherein eIF-4E sequestering agent comprises a sequence having an amino acid sequence selected from YxxxxLφ, Yx+xfφφ, +φxYx+xfφφ, +φφY−+xF/AφφxxRxSP, and +φφY−+xfLφxxRxSP, or +φxYx+xfLφxxxxxx wherein + and − refer to a charged amino acid; φ is a hydrophobic amino acid; x is any amino acid; and the capital letters refer to the known one letter code for amino acids

14. The method of claim 13, wherein said sequestering agent is selected from 4E-BP, eIF-4G, p82, p150, p130 and p20.

15. The method of claim 14, wherein said sequestering agent is selected from 4E-BP1, 4E-BP2, 4E-BP3, eIF-4G1, eIF-4-G2, 4E-BP, and eIF-4G.

16. The method of claim 15, wherein said sequestering agent is selected from 4E-BP1, 4E-BP2, 4E-BP3, eIF-4G1, eIF-4G2, 4E-BP, and eIF-4G.

17. Method of identifying an agent which modulates glucose and/or fat metabolism in vivo comprising:
a) providing a translationally active preparation of translation factors and at least one mRNA having a cap structure whose translation is cap-dependent;
b) measuring the initiation of translation on said mRNA, or the binding of at least some translation factors of a) to said cap of said mRNA in the presence and in the absence of an agent suspected of modulating the translation efficiency of cap-dependent mRNAs or the binding of translation factors to the cap structure thereof, thereby identifying an agent which modulates cap-dependent translation and wherein a difference in the translation activity and/or binding in the presence of the agent, as compared to that in the absence thereof identifies said agent as a modulator of cap-dependent translation;
c) administering said agent identified in b) to an animal; and
d) measuring glucose and/or lipid levels in the animal of step c) and comparing same with that of a control animal, not having been administered said agent, wherein a difference in glucose and/or lipid levels of the animal of step c) as compared to that of the control animal identifies said agent as a modulator of glucose or fat metabolism in vivo.

18. The method of claim 17, where the agent is obtained from a library of compounds.

19. The method of claim 18, wherein the animal is a mammal.

20. The method of claim 19, wherein said mammal is a mouse or human.

21. A modulator of glucose or fat metabolism in vivo identified by any one of the methods of claims 17, 18, 19 or 20.

22. A method of decreasing fat tissue growth and/or weight gain, comprising:
a) administering an agent which desequesters eIF-4E from a sequestering agent, thereby increasing the amount of eIF-4E available for a formation of eIF-4F preinitiation complex, leading to an increase of translation of cap-dependent mRNAs implicated in a reduction of a tissue growth and/or weight gain.

23. The method of claim 22, wherein said sequestration of eIF-4E is through its interaction with 4E-BP1.

24. The method of claim 23, wherein said desequestration of eIF-4E from 4E-BP1 is effected by an antibody specific to the eIF-4E interaction domain of 4E-BP1, or an epitope-bearing portion thereof.

25. The method of claim 24, wherein said desequestration or said inhibition of said sequestration is effected by an inhibition of the synthesis of 4E-BP1.

26. The method of claim 25, comprising an agent which inhibits the synthesis of 4E-BP1, wherein said agent comprises an antisense RNA complementary to the nucleotide sequence encoding for 4E-BP1.

27. A method of determining whether an agent modulates fat tissue growth and/or weight gain in an animal comprising:
a) providing a translationally active preparation of translation factors and at least one mRNA having a cap structure whose translation is cap-dependent;
b) measuring the initiation of translation on said mRNA, or the binding of at least some translation factors of a) to said cap of said mRNA in the presence and in the absence of an agent suspected of modulating the translation efficiency of cap-dependent mRNAs or the binding of translation factors to the cap structure thereof, thereby identifying an agent which modulates cap-dependent translation and wherein a difference in the translation activity and/or binding in the presence of the agent, as compared to that in the absence thereof identifies said agent as a modulator of cap-dependent translation;
c) administering said agent identified in b) to an animal; and
d) measuring fat tissue growth and/or weight gain in the animal of step c) and comparing same with that of a control animal, not having been administered said agent, wherein a difference in fat tissue growth and/or weight gain of the animal of step c) as compared to that of the control animal identifies said agent as a modulator of fat tissue growth and/or weight gain in vivo.

28. The method of claim 27, where the agent is obtained from a library of compounds.

29. The method of claim 28, wherein the animal is a mammal.

30. The method of claim 29, wherein said mammal is a mouse or human.

31. A modulator of glucose or fat metabolism in vivo identified by any one of the methods of claims 28, 29, 30 or 31.

32. A method of treating obesity, comprising administering to an obese animal, or an animal susceptible of becoming obese, an agent which increases the amount of eIF-4E available for a formation of eIF-4F preinitiation complex.

33. The method of claim 32, wherein said agent is an agent which desequesters eIF-4E from 4E-BP1.

34. A method of determining whether an agent modulates obesity in an animal comprising:
a) providing a translationally active preparation of translation factors and at least one mRNA having a cap structure whose translation is cap-dependent;
b) measuring the initiation of translation on said mRNA, or the binding of at least some translation factors of a) to said cap of said mRNA in the presence and in the absence of an agent suspected of modulating the translation efficiency of cap-dependent mRNAs or the binding of translation factors to the cap structure thereof, thereby identifying an agent which modulates cap-dependent translation and wherein a difference in the translation activity and/or binding in the presence of the agent, as compared to that in the absence thereof identifies said agent as a modulator of cap-dependent translation;
c) administering said agent identified in b) to an animal; and
d) assessing obesity in the animal of step c) and comparing same with that of a control animal, not having been administered said agent, wherein a difference in obesity of the animal of step c) as compared to that of the control animal identifies said agent as a modulator of obesity in vivo.

35. The method of claim 18, where the agent is obtained from a library of compounds.

36. The method of claim 35, wherein the animal is a mammal.

37. The method of claim 36, wherein said mammal is a mouse or human.

38. A modulator of glucose and/or fat metabolism in vivo identified by any one of the methods of claims 7, 17, 22, 27, 34, 35 or 36.

39. A method of treating diabetes type II and associated complications, comprising administering to an animal suffering from type II diabetes, or at risk of suffering therefrom, an agent which increases the amount of eIF-4E available for a formation of eIF-4F preinitiation complex.

40. The method of claim 39, wherein said agent is an agent which desequesters eIF-4E from 4E-BP1.

41. A method of identifying an agent which modulates glucose and/or fat metabolism in vivo comprising:
a) incubating a portion of eIF-4E capable of directly binding with a peptide comprising an eIF-4E-binding domain;
b) assessing said direct binding between said portion of eIF-4E and said peptide in a presence and in an absence of an agent wherein an agent which potentially modulates glucose and/or fat metabolism is identified when a difference in said binding in the presence of said agent, as compared to that in the absence thereof is observed;
c) administering said agent identified in b) to an animal; and
d) measuring glucose and/or lipid levels in the animal of step c) and comparing same with that of a control animal, not having been administered said agent, wherein a difference in glucose and/or lipid levels of the animal of step c) as compared to that of the control animal identifies said agent as a modulator of glucose or fat metabolism in vivo.
